(19) United States
(12) Patent Application Publication
(10) Pub. No.: US 2018/0078605 A1
(43) Pub. Date: Mar. 22, 2018

(54) PERIPHERAL KAPPA OPIOID RECEPTOR AGONISTS FOR UREMIC PRURITUS IN DIALYSIS PATIENTS

(71) Applicant: Cara Therapeutics, Inc., Stamford, CT (US)

(72) Inventors: Robert H. SPENCER, New Hope, PA (US); Frédérique MENZAGHI, Rye, NY (US); Derek T. CHALMERS, Riverside, CT (US)

(73) Assignee: Cara Therapeutics, Inc., Stamford, CT (US)

(21) Appl. No.: 15/811,199

(22) Filed: Nov. 13, 2017

Related U.S. Application Data
(63) Continuation-in-part of application No. 15/193,837, filed on Jun. 27, 2016, now abandoned.
Application No. 15/193,837 is a continuation-in-part of application No. 15/137,033, filed on Apr. 25, 2016, now abandoned.
Application No. 15/137,033 is a continuation of application No. 14/557,576, filed on Dec. 2, 2014, now Pat. No. 9,321,810.
Application No. 14/557,576 is a continuation of application No. 12/868,790, filed on Aug. 26, 2010, now Pat. No. 8,906,859.
Application No. 12/868,790 is a continuation-in-part of application No. 12/480,059, filed on Jun. 8, 2009, now Pat. No. 8,236,766.
Application No. 12/480,059 is a continuation-in-part of application No. 12/119,311, filed on May 12, 2008, now Pat. No. 7,713,937.
Application No. 12/119,311 is a continuation-in-part of application No. 11/938,771, filed on Nov. 12, 2007, now Pat. No. 7,402,564.
Application No. 11/938,771 is a continuation of application No. 11/938,776, filed on Nov. 12, 2007, now Pat. No. 7,842,662.
(60) Provisional application No. 62/188,189, filed on Jul. 2, 2015.
(60) Provisional application No. 62/195,857, filed on Jul. 23, 2015.
(60) Provisional application No. 60/928,550, filed on May 10, 2007.
(60) Provisional application No. 60/858,109, filed on Nov. 10, 2006.
(60) Provisional application No. 60/928,551, filed on May 10, 2007.
(60) Provisional application No. 60/928,557, filed on May 10, 2007.
(60) Provisional application No. 60/928,527, filed on May 10, 2007.
(60) Provisional application No. 60/858,120, filed on Nov. 10, 2006.

(51) Int. Cl. A61K 38/07; C07K 7/02; C07K 5/107; A61K 38/08; C07K 5/11; C07K 5/103

(52) U.S. Cl. CPC A61K 38/07; C07K 7/02; C07K 5/1016; C07K 5/101; A61K 38/08; C07K 5/1019

(57) ABSTRACT

The invention provides a method of prevention, inhibition or treatment of uremic pruritus in a dialysis patient by administering an effective amount of a kappa opioid receptor agonist. Also provided is a method of inhibition or treatment of adverse symptoms associated with dialysis affecting the quality of life of dialysis patient, the method includes administering an effective amount of a kappa opioid receptor agonist. The adverse symptoms associated with dialysis addressable by the methods of the invention include uremic pruritus, sleep disruption, depression and other mood alterations.

### FIELD OF THE INVENTION

The invention relates to methods for the use of synthetic peptide amides incorporating D-amino acids in the peptide chain and more particularly to methods for the use of such synthetic peptide amides that are kappa opioid receptor agonists, and methods for their use as prophylactic and therapeutic agents.

### BACKGROUND

Kappa opioid receptors have been suggested as targets for intervention for treatment or prevention of a wide array of diseases and conditions by administration of kappa opioid receptor agonists. See for example, Jolivalt et al., Diabetologia, 49(11):2775-85; Epub Aug. 19, 2006), describing efficacy of asimadoline, a kappa receptor agonist in rodent diabetic neuropathy; and Bileviciute-Ljungar et al., Eur. J. Pharm. 494:139-46 (2004) describing the efficacy of kappa agonist U-50,488 in the rat chronic constriction injury (CCI) model of neuropathic pain and the blocking of its effects by the opioid antagonist, naloxone. These observations support the use of kappa opioid receptor agonists for treatment of diabetic, viral and chemotherapy-induced neuropathic pain. The use of kappa receptor agonists for treatment or prevention of visceral pain including gynecological conditions such as dysmenorrheal cramps and endometriosis has also been reviewed. See for instance, Riviere, Br. J. Pharmacol. 141:1331-4 (2004).

Kappa opioid receptor agonists have also been proposed for the treatment of pain, including hyperalgesia. Hyperalgesia is believed to be caused by changes in the milieu of the peripheral sensory terminal occur secondary to local tissue damage. Tissue damage (e.g., abrasions, burns) and inflammation can produce significant increases in the excitability of polymodal nociceptors (C fibers) and high threshold mechanoreceptors (Handwerker et al. (1991) Proceeding of the VIth World Congress on Pain, Bond et al., eds., Elsevier Science Publishers BV, pp. 59-70; Schaible et al. (1993) Pain 55:5-54). This increased excitability and exaggerated responses of sensory afferents is believed to underlie hyperalgesia, where the pain response is the result of an exaggerated response to a stimulus. The importance of hyperalgesia in the post-injury pain state has been repeatedly demonstrated and appears to account for a major proportion of pain experienced in the post-injury/inflammatory state. See for example, Woold et al. (1993) Anesthesia and Analgesia 77:362-79; Dubner et al. (1994) In, Textbook of Pain, Melzack et al., eds., Churchill-Livingstone, London, pp. 225-242.

Kappa opioid receptors have been suggested as targets for the prevention and treatment of cardiovascular disease. See for example, Wu et al. “Cardioprotection of Preconditioning by Metabolic Inhibition in the Rat Ventricular Myocyte—Involvement of kappa Opioid Receptor” (1999) Circulation Res vol. 84: pp. 1388-1395. See also Yu et al. “Anti-Arrhythmic Effect of Kappa Opioid Receptor Stimulation in the Perfused Rat Heart: Involvement of a cAMP-Dependent Pathway” (1999) J Mol Cell Cardiol. vol. 31(10): pp. 1809-1819.

It has also been found that development or progression of these diseases and conditions involving neurodegeneration or neuronal cell death can be prevented, or at least slowed, by treatment with kappa opioid receptor agonists. This improved outcome is believed to be due to neuroprotection by the kappa opioid receptor agonists. See for instance, Kaushik et al. “Neuroprotection in Glaucoma” (2003) J. Postgraduate Medicine vol. 49 (1): pp. 90-95.

The presence of kappa opioid receptors on immune cells (Bidlak et al., (2000) Clin. Diag. Lab. Immunol. 7(5):719-723) has been implicated in the inhibitory action of a kappa opioid receptor agonist, which has been shown to suppress HIV-1 expression. See Peterson P K et al., Biochem Pharmacol. 2001, 61(19):1145-51.

Walker, Adv. Exp. Med. Biol. 521:148-60 (2003) appraised the anti-inflammatory properties of kappa agonists for treatment of osteoarthritis, rheumatoid arthritis, inflammatory bowel disease and eczema. Bileviciute-Ljungar et al., Rheumatology 45:295-302 (2006) describe the reduction of pain and degeneration in Freund's adjuvant-induced arthritis by the kappa agonist U-50,488.

Pruritus (herein interchangeably referred to as “pruritis”), or itching, occurs in many diseases and conditions such as, for instance, ocular pruritus associated with conjunctivitis, as well as pruritus associated with dermatological conditions such as eczema (dermatitis), including atopic or contact dermatitis, psoriasis, polycythemia vera, lichen planus, lichen simplex chronicus, pediculosis (lice), thyrotoxicosis, tinea pedis, urticaria, scabies, vaginitis, anal pruritus associated with hemorrhoids, as well as insect-bite pruritus and drug-induced pruritus, such as pruritus induced by mu opioids, including morphine. Pruritus is also associated with chronic kidney dysfunction, including end-stage renal disease, where many patients are receiving kidney dialysis, and other forms of cholestasis, including primary biliary cirrhosis, intrahepatic cholestasis of pregnancy, chronic cholestatic liver disease, uremia, malignant cholestasis, and jaundice.

Uremic pruritus, also called chronic kidney disease-associated pruritus, is common in patients suffering from chronic kidney dysfunction (CKD), occurring in about 20%-50% of patients with renal failure. Uremic pruritus is a chronic itching condition causing long-term pain and suffering, especially in patients with advanced or end-stage renal disease. Currently, this condition is managed by optimizing regimens to provide adequate dialysis of the patient's blood. Topical emollients are used in patients with localized itching and antihistamines delivered orally have been found to provide some, though limited benefits in dialysis patients. Currently, the only cure is a kidney transplant which is not available to most patients due to limited organ availability, tissue matching requirements and the high costs of surgery and post-surgical therapy.

Wikstrom et al., J. Am. Soc. Nephrol. 16:3742-7 (2005) describes the use of the kappa agonist, TRK-820 for treatment of uremic and opiate-induced pruritis, and Ko et al., J. Pharmacol. Exp. Ther. 305:173-9 (2003) describe the efficacy of U-50,488 in morphine-induced pruritis in the monkey.

Application of peripheral opioids including kappa agonists for treatment of gastrointestinal diseases has also been extensively reviewed. See for example, Lembo, Diges. Dis. 24:91-8 (2006) for a discussion of use of opioids in treatment of digestive disorders, including irritable bowel syndrome (IBS), ileus, and functional dyspepsia.

Ophthalmic disorders, including ocular inflammation and glaucoma have also been shown to be addressable by kappa opioids. See Potter et al., J. Pharmacol. Exp. Ther. 309:548-53 (2004), describing the role of the potent kappa opioid receptor agonist, bremazocine, in reduction of intraocular pressure and blocking of this effect by norbinaltorphimine (norBNI), the prototypical kappa opioid receptor antagonist; and Dortch-Carnes et al., CNS Drug Rev. 11(2):195-212 (2005). U.S. Pat. No. 6,191,126 to Gamache discloses the use of kappa opioid agonists to treat ocular pain. Otic pain has also been shown to be treatable by administration of kappa opioid agonists. See U.S. Pat. No. 6,174,878 also to Gamache.

Kappa opioid agonists increase the renal excretion of water and decrease urinary sodium excretion (i.e., produces a selective water diuresis, also referred to as aquaresis). Many, but not all, investigators attribute this effect to a suppression of vasopressin secretion from the pituitary. Studies comparing centrally acting and purportedly peripherally selective kappa opioids have led to the conclusion that kappa opioid receptors within the blood-brain barrier are responsible for mediating this effect. Other investigators have proposed to treat hyponatremia with nociceptin peptides or charged peptide conjugates that act peripherally at the nociceptin receptor, which is related to but distinct from the kappa opioid receptor (D. R. Kapusta, Life Sci., 60:15-21, 1997) (U.S. Pat. No. 5,840,696). U.S. Pat Appl. 20060052284.

### SUMMARY OF THE INVENTION

The invention provides methods of use of a selective kappa opioid receptor agonist (interchangeably referred to herein as a kappa receptor agonist or simply as a kappa agonist) which is a synthetic peptide amide of the invention, as described below.

The invention also provides methods of use of

a pharmaceutical composition, which includes a synthetic peptide amide of the invention and a pharmaceutically acceptable diluent, excipient or carrier.

Also provided is a method of treating or preventing a kappa opioid receptor-associated disease or condition in a mammal. The method includes administering to the mammal a composition that includes an effective amount of a synthetic peptide amide of the invention. The invention also provides uses of the synthetic peptide amides of the invention for the preparation of medicaments and pharmaceutical compositions useful for the treatment of a kappa opioid receptor-associated disease or condition in a mammal.

The invention further provides a method of prophylaxis or treatment of a kappa opioid receptor-associated disease or condition in a mammal, wherein a synthetic peptide amide of the invention is co-administered with a reduced dose of a mu opioid agonist analgesic compound to produce a therapeutic analgesic effect, the mu opioid agonist analgesic compound having an associated side effect, (especially respiratory depression, sedation, euphoria, antidiuresis, nausea, vomiting, constipation, and physical tolerance, dependence, and addiction). The reduced dose of the mu opioid agonist analgesic compound administered by this method has lower associated side effects than the side effects associated with the dose of the compound necessary to achieve the same therapeutic analgesic effect when administered alone.

The invention also provides a method of treating or preventing peripheral hyperalgesia, wherein the method includes topically applying or locally administering to a mammal in need of the treatment, an effective amount of a composition that includes an anti-hyperalgesically-effective amount of a synthetic peptide amide of the invention in a vehicle formulated for topical application or local administration.

The invention also provides a method of treating or preventing hyponatremia or hypokalemia, and thereby treating or preventing a disease or condition associated with hyponatremia or hypokalemia, such as congestive heart failure, liver cirrhosis, nephrotic syndrome, hypertension, or edema, and preferably where increased vasopres sin secretion is associated with said disease or disorder, wherein the method includes administering to a mammal an aquaretically effective amount of a synthetic peptide amide of the invention in a pharmaceutically acceptable diluent, excipient or carrier.

The present invention provides a method of treatment of a patient suffering from uremic pruritus including administering an effective amount of a kappa opioid receptor agonist to a patient undergoing a haemodialysis regimen on at least one of the days in which the dialysis procedure occurs. The invention also provides a method of reducing an adverse symptom associated with dialysis in a patient undergoing a haemodialysis regimen, including administering an effective amount of a kappa opioid receptor agonist.

Related U.S. Pat. Nos. 7,402,564; 7,713,937 and 7,842,662 the disclosures of which are herein incorporated by reference, provide further information useful in the practice of the present invention.

### BRIEF DESCRIPTION OF THE FIGURES

FIG. 1 is a schematic of the study design of Part A of the clinical trial of CR845 in patients with uremic pruritus.

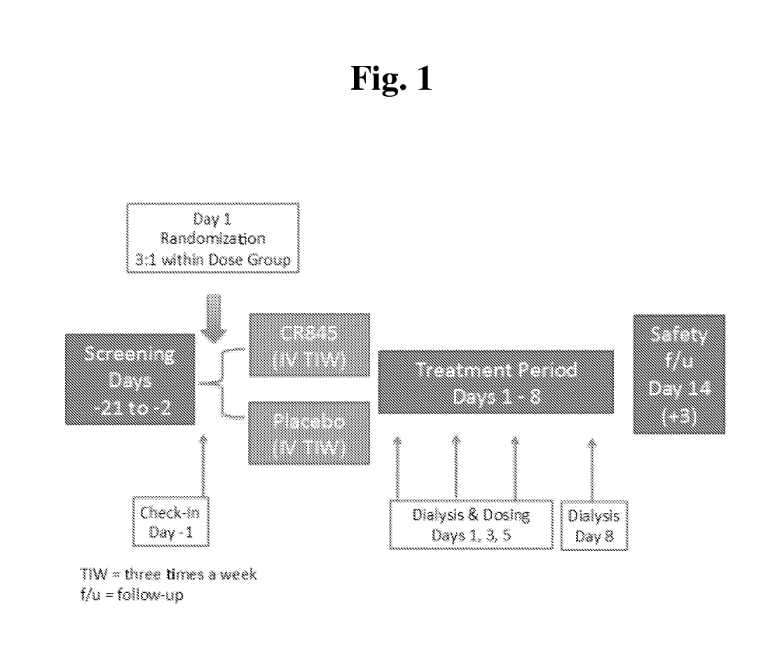

FIG. 2 is a schematic of the study design of Part B of the clinical trial of CR845 in patients with uremic pruritus.

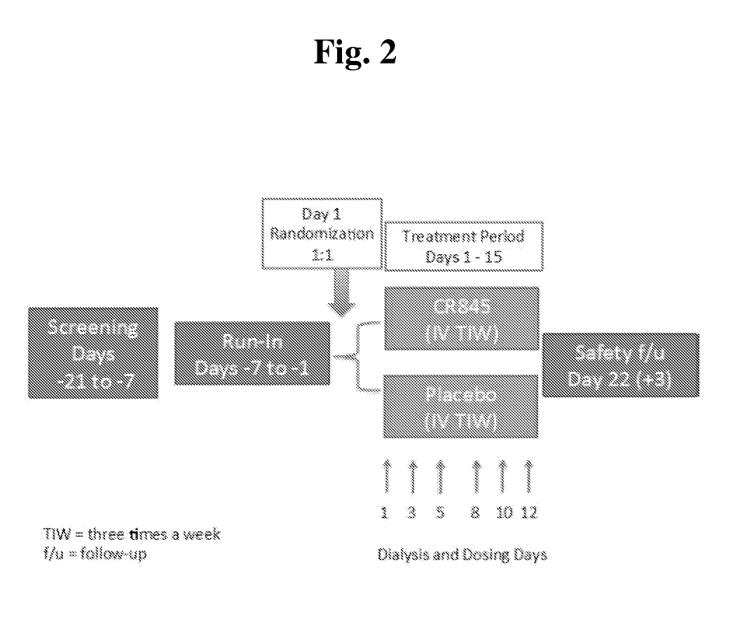

FIG. 3 shows the pharmacokinetics of CR845 in nineteen dialysis patients suffering from end stage renal disease (ESRD), exhibiting a half-life of ˜24 hrs. CR845 levels in dialysis patient blood is plotted at 5, 15 and 30 minutes, 1, 2, 4 6, 8 12 and 24 hours after post-dialysis administration of CR845. CR845 levels at day 3 and at day 5 pre-dialysis and post-dialysis before administration of CR845 are interpolated between days 1 and 5.

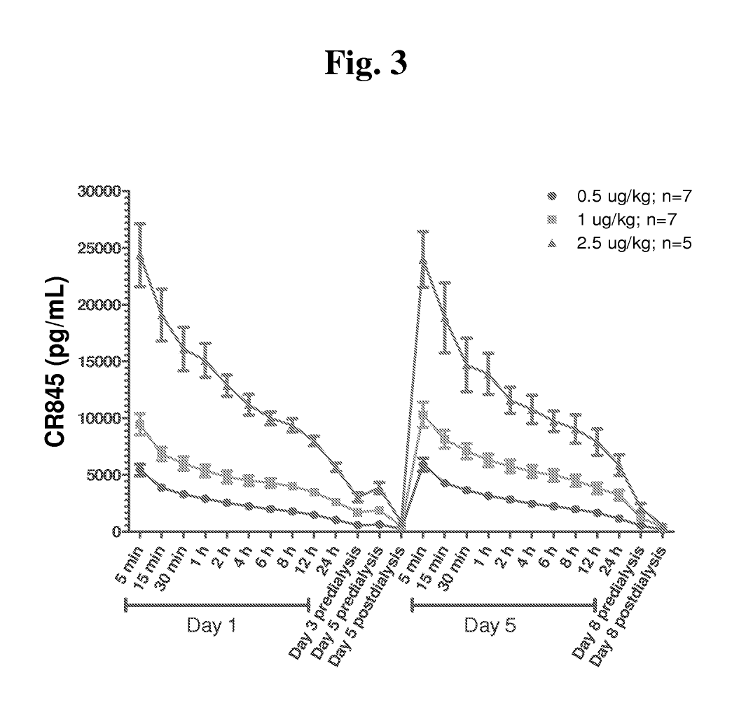

FIG. 4 is a histogram comparing change in itch from baseline on a visual analog scale (VAS) in placebo-treated and CR845-treated dialysis patients with end stage renal disease (ESRD). Bars represent standard error about the mean (S.E.M.).

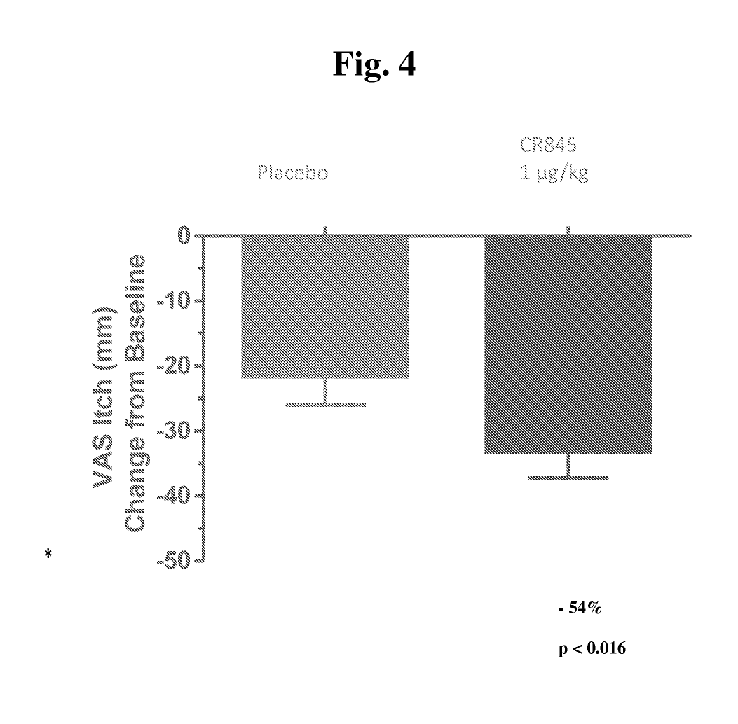

FIG. 5 is a histogram comparing change in itch in placebo-treated and CR845-treated dialysis patients with end stage renal disease (ESRD) during the run-in period, at week 1 and week 2 of the CR845 treatment trial. Bars represent standard error about the mean (S.E.M.).

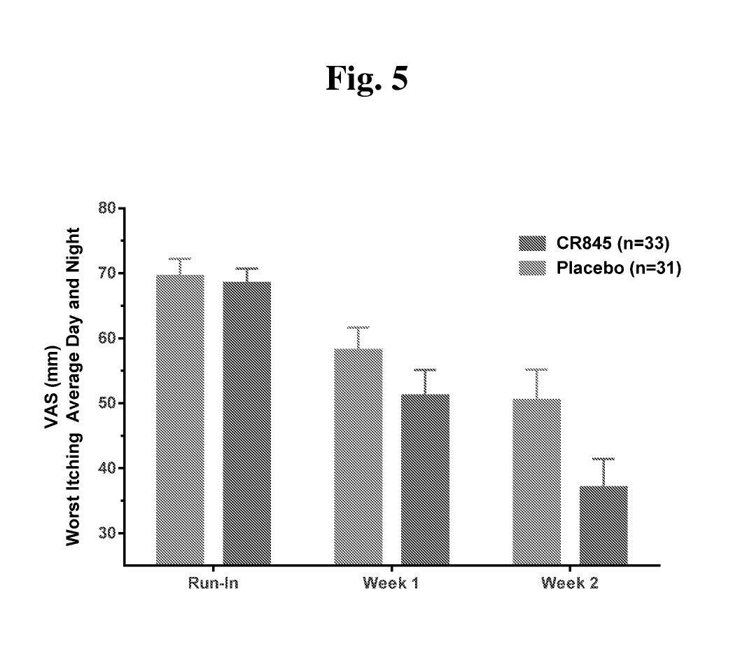

FIG. 6 shows the progress of the reduction in “worst itching” average over daytime and night time as assessed by a visual analog scale (VAS) in CR845-treated dialysis patients over the 15 days of the trial. Bars represent standard error about the mean (S.E.M.).

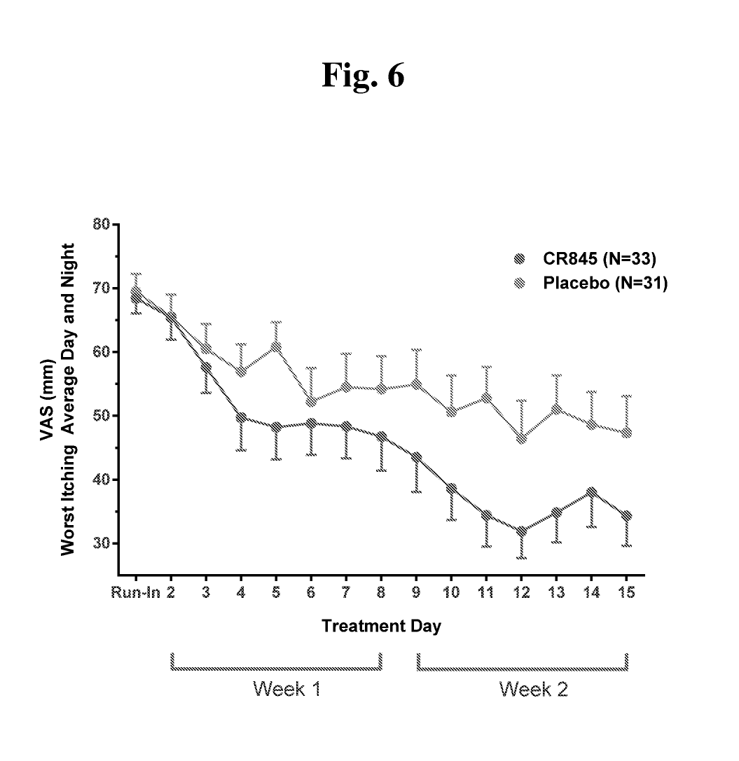

FIGS. 7A and 7B show the significant reduction in Worst itch intensity reported for day time and night time, respectively in CR845-treated patients by the second week of the trial. The reductions in levels of itch in CR845-treated patients assessed on a visual analog scale (VAS) is significant (p<0.05) in the daytime and highly significant (p<0.01) in the night time by the second week of the trial. Bars represent standard error about the mean (S.E.M.).

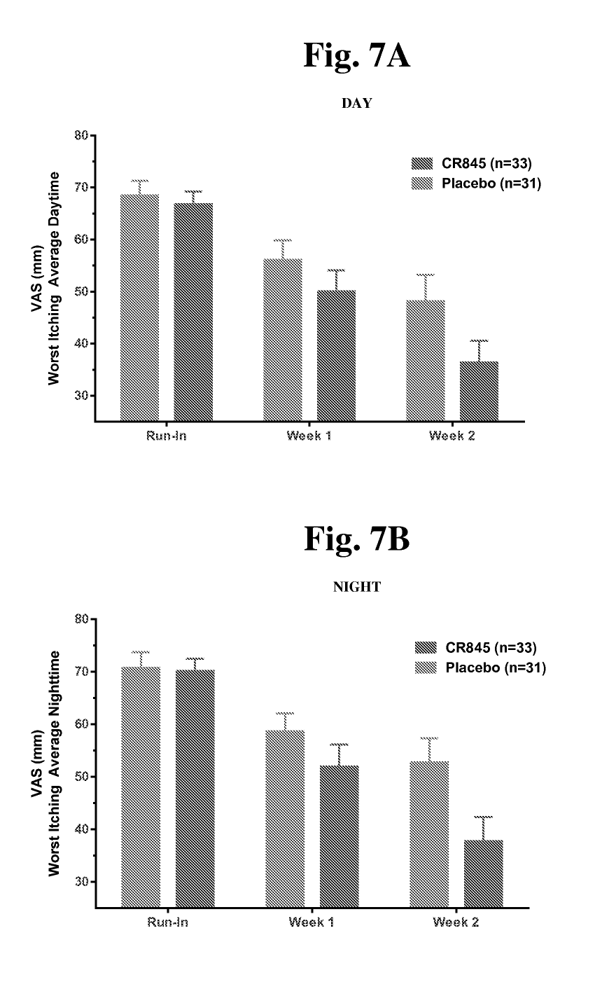

FIG. 8 is a histogram comparing change in itch-related quality of life assessed as the Skindex-10 score in placebo-treated and CR845-treated dialysis patients. Bars represent standard error about the mean (S.E.M.).

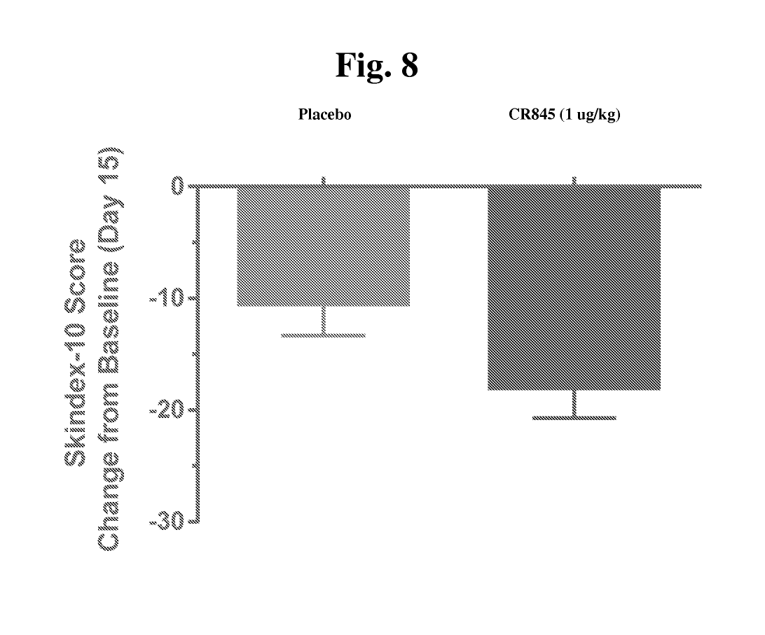

FIG. 9 shows the improvement in Skindex-10 (reduction is an improvement on this scale) in itch-related quality of life scores in placebo and CR845-treated dialysis patients. Bars represent standard error about the mean (S.E.M.).

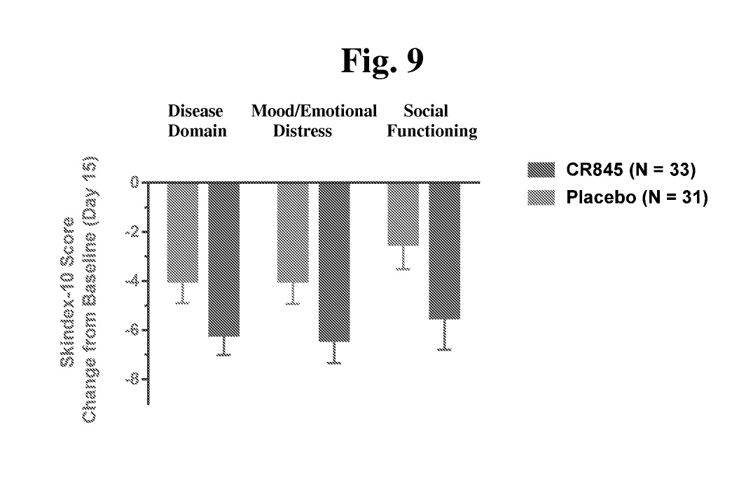

FIG. 10 shows the reduction in itch-related sleep disturbances assessed on the itch MOS Sleep problems Index II (SLP-9). Bars represent standard error about the mean (S.E.M.).

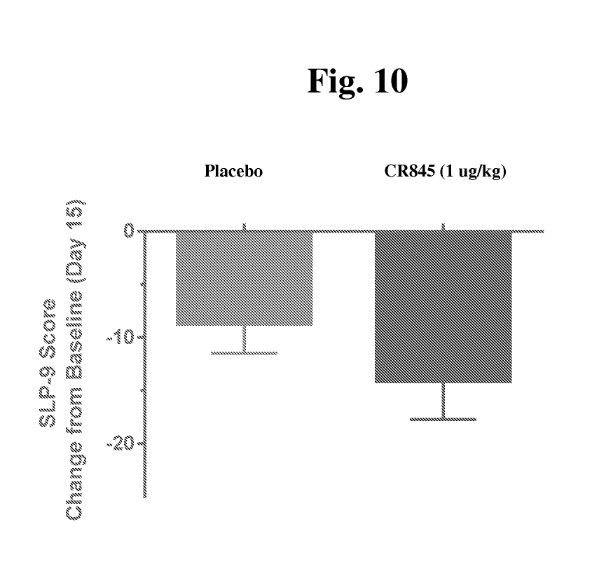

### DETAILED DESCRIPTION

As used throughout this specification, the term “synthetic peptide amide” means a compound of the invention conforming to formula I as described in U.S. Pat. No. 7,402,564, or a stereoisomer, mixture of stereoisomers, prodrug, pharmaceutically acceptable salt, hydrate, solvate, acid salt hydrate, N-oxide or isomorphic crystalline form thereof.

In one embodiment the synthetic peptide amide useful in the practice of the invention exhibits a long lasting duration of action in a mammal, such as a human. In one aspect, the synthetic peptide amide has a duration of action that is at least about 50% of maximum efficacy at three hours post administration of 0.1 mg/kg of the synthetic peptide amide. In another aspect the synthetic peptide amide has a duration of action that is at least about 75% of maximum efficacy at three hours post administration of 0.1 mg/kg of the synthetic peptide amide. In a particular aspect the synthetic peptide amide has a duration of action that is at least about 90% of maximum efficacy at 3 hrs post administration of 0.1 mg/kg of the synthetic peptide amide. In a specific aspect, the synthetic peptide amide has a duration of action that is at least about 95% of maximum efficacy at three hours post administration of 0.1 mg/kg of the synthetic peptide amide.

In another embodiment, the invention provides a pharmaceutical composition that includes therapeutic uses of a synthetic peptide amide according to any of the above embodiments and a pharmaceutically acceptable excipient or carrier. The invention provides methods, compositions, or dosage forms that employ and/or contain synthetic peptide amides of the invention that are selective for the kappa opioid receptor. In particular embodiments, the synthetic peptide amides of the invention exhibit a strong affinity for the kappa opioid receptor and have a high potency as kappa opioid receptor agonists.

A pro-drug of a compound such as the synthetic peptide amides of the invention include pharmaceutically acceptable derivatives which upon administration can convert through metabolism or other process to a biologically active form of the compound. Pro-drugs are particularly desirable where the pro-drug has more favorable properties than does the active compound with respect to bioavailability, stability or suitability for a particular formulation.

As used herein, a kappa opioid receptor-associated disease, condition or disorder is any disease, condition or disorder that is preventable or treatable by activation of a kappa opioid receptor. In one aspect, the synthetic peptide amides of the invention are kappa opioid receptor agonists that activate the kappa opioid receptor. In some embodiments, a particular dose and route of administration of the synthetic peptide amide of the invention can be chosen by a clinician to completely prevent or cure the disease, condition or disorder. In other embodiments a particular dose and route of administration of the synthetic peptide amide of the invention chosen by the clinician ameliorates or reduces one or more symptoms of the disease, condition or disorder.

As used herein, “effective amount” or “sufficient amount” of the synthetic peptide amide of the invention refers to an amount of the compound as described herein that may be therapeutically effective to inhibit, prevent, or treat a symptom of a particular disease, disorder, condition, or side effect. As used herein, a “reduced dose” of a mu opioid agonist analgesic compound refers to a dose which when used in combination with a kappa opioid agonist, such as a synthetic peptide amide of the invention, is lower than would be ordinarily provided to a particular patient, for the purpose of reducing one or more side effects of the compound. The dose reduction can be chosen such that the decrease in the analgesic or other therapeutic effect of the compound is an acceptable compromise in view of the reduced side effect(s), where the decrease in analgesic or other therapeutic effects of the mu opioid agonist analgesic are wholly or at least partially offset by the analgesic or other therapeutic effect of the synthetic peptide amide of the invention. Co-administration of a mu opioid agonist analgesic compound with a synthetic peptide amide of the invention which acts as a kappa opioid agonist also permits incorporation of a reduced dose of the synthetic peptide amide and/or the mu opioid agonist analgesic compound to achieve the same therapeutic effect as a higher dose of the synthetic peptide amide or the mu opioid agonist analgesic compound if administered alone.

As used herein, “pharmaceutically acceptable” refers to compounds, materials, compositions, and/or dosage forms which are, within the scope of sound medical judgment, suitable for contact with the tissues of human beings and animals without severe toxicity, irritation, allergic response, or other complications, commensurate with a benefit-to-risk ratio that is reasonable for the medical condition being treated.

As used herein, “dosage unit” refers to a physically discrete unit suited as unitary dosages for a particular individual or condition to be treated. Each unit may contain a predetermined quantity of active synthetic peptide amide compound(s) calculated to produce the desired therapeutic effect(s), optionally in association with a pharmaceutical carrier. The specification for the dosage unit forms may be dictated by (a) the unique characteristics of the active compound or compounds, and the particular therapeutic effect to be achieved, and (b) the limitations inherent in the art of compounding such active compound or compounds. The dosage unit is often expressed as weight of compound per unit body weight, for instance, in milligrams of compound per kilogram of body weight of the subject or patient (mg/kg). Alternatively, the dosage can be expressed as the amount of the compound per unit body weight per unit time, (mg/kg/day) in a particular dosage regimen. In a further alternative, the dosage can be expressed as the amount of compound per unit body surface area (mg/m²) or per unit body surface area per unit time (mg/m²/day). For topical formulations, the dosage can be expressed in a manner that is conventional for that formulation, e.g., a one-half inch ribbon of ointment applied to the eye, where the concentration of compound in the formulation is expressed as a percentage of the formulation.

As used herein, a “pharmaceutically acceptable salt” refers to a derivative of a compound wherein the parent compound is modified by making an acid or a base salt thereof. Examples of pharmaceutically acceptable salts include, but are not limited to, mineral or organic acid salts of basic residues such as amines; alkali or organic salts of acidic residues such as carboxylic acids and the like. The pharmaceutically acceptable salts include the conventional non-toxic salts or the quaternary ammonium salts of the parent compound formed, for example, from non-toxic inorganic or organic acids. For instance, such conventional non-toxic salts include those derived from inorganic acids such as hydrochloric, hydrobromic, sulfuric, sulfamic, phosphoric, nitric acids and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acids, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Thus, a pharmaceutically acceptable salt of a synthetic peptide amide can be formed from any such peptide amide having either acidic, basic or both functional groups. For example, a peptide amide having a carboxylic acid group, may in the presence of a pharmaceutically suitable base, form a carboxylate anion paired with a cation such as a sodium or potassium cation. Similarly, a peptide amide having an amine functional group may, in the presence of a pharmaceutically suitable acid such as HCl, form a salt.

An example of a pharmaceutically acceptable solvate of a synthetic peptide amide is a combination of a peptide amide with solvent molecules which yields a complex of such solvent molecules in association with the peptide amide. Combinations of a drug and propylene glycol (1,2-propanediol) have been used to form pharmaceutical drug solvates. See for example U.S. Pat. No. 3,970,651. Other suitable solvates are hydrates of drug compounds. Such hydrates include hydrates which either have comparable activity or hydrates which are converted back to the active compound following administration. A pharmaceutically acceptable N-oxide of a synthetic peptide amide is such a compound that contains an amine group wherein the nitrogen of the amine is bonded to an oxygen atom.

A pharmaceutically acceptable crystalline, isomorphic crystalline or amorphous form of a synthetic peptide amide of the invention can be any crystalline or non-crystalline form of a pharmaceutically acceptable acidic, basic, zwitterionic, salt, hydrate or any other suitably stable, physiologically compatible form of the synthetic peptide amide according to the invention.

The synthetic peptide amides useful in the practice of the invention can be incorporated into pharmaceutical compositions. The compositions can include an effective amount of the synthetic peptide amide in a pharmaceutically acceptable diluent, excipient or carrier. Conventional excipients, carriers and/or diluents for use in pharmaceutical compositions are generally inert and make up the bulk of the preparation.

In a particular embodiment, the synthetic peptide amide useful in the practice of the invention is a kappa opioid receptor agonist. In another embodiment, the synthetic peptide amide is a selective kappa opioid receptor agonist. The target site can be a kappa receptor in the patient or subject in need of such treatment or prophylaxis. Certain synthetic peptide amide kappa opioid receptor agonists of the invention are peripherally acting and show little or no CNS effects at therapeutically effective doses.

The pharmaceutical excipient or carrier can be any compatible, non-toxic substance suitable as a vehicle for delivery the synthetic peptide amide of the invention. Suitable excipients or carriers include, but are not limited to, sterile water (preferably pyrogen-free), saline, phosphate-buffered saline (PBS), water/ethanol, water/glycerol, water/sorbitol, water/polyethylene glycol, propylene glycol, cetylstearyl alcohol, carboxymethylcellulose, corn starch, lactose, glucose, microcrystalline cellulose, magnesium stearate, polyvinylpyrrolidone (PVP), citric acid, tartaric acid, oils, fatty substances, waxes or suitable mixtures of any of the foregoing.

The pharmaceutical composition useful in the practice of to the invention can be formulated as a liquid, semisolid or solid dosage form. For example the pharmaceutical preparation can be in the form of a solution for injection, drops, syrup, spray, suspension, tablet, patch, capsule, dressing, suppository, ointment, cream, lotion, gel, emulsion, aerosol or in a particulate form, such as pellets or granules, optionally pressed into tablets or lozenges, packaged in capsules or suspended in a liquid. The tablets can contain binders, lubricants, diluents, coloring agents, flavoring agents, wetting agents and may be enteric-coated to survive the acid environment of the stomach and dissolve in the more alkaline conditions of the intestinal lumen. Alternatively, the tablets can be sugar-coated or film coated with a water-soluble film. Pharmaceutically acceptable adjuvants, buffering agents, dispersing agents, and the like, may also be incorporated into the pharmaceutical compositions.

Binders include for instance, starch, mucilage, gelatin and sucrose. Lubricants include talc, lycopodium, magnesium and calcium stearate/stearic acid. Diluents include lactose, sucrose, mannitol, salt, starch and kaolin. Wetting agents include propylene glycol and sorbitan monostearate.

As used herein, local application or administration refers to administration of a pharmaceutical preparation according to the invention to the site, such as an inflamed joint, that exhibits the painful and/or inflamed condition. Such local application includes intrajoint, such as intra-articular application, via injection, application via catheter or delivery as part of a biocompatible device. Thus, local application refers to application to a discrete internal area of the body, such as, for example, a joint, soft tissue area (such as muscle, tendon, ligaments, intraocular or other fleshy internal areas), or other internal area of the body. In particular, as used herein, local application refers to applications that provide substantially no systemic delivery and/or systemic administration of the active agents in the present compositions. Also, as used herein, local application is intended to refer to applications to discrete areas of the body, that is, other than the various large body cavities (such as, for example, the peritoneal and/or pleural cavities).

As used herein, topical application refers to application to the surface of the body, such as to the skin, eyes, mucosa and lips, which can be in or on any part of the body, including but not limited to the epidermis, any other dermis, or any other body tissue. Topical administration or application means the direct contact of the pharmaceutical preparation according to the invention with tissue, such as skin or membrane, particularly the cornea, or oral, vaginal or anorectal mucosa. Thus, for purposes herein topical application refers to application to the tissue of an accessible body surface, such as, for example, the skin (the outer integument or covering) and the mucosa (the mucus-producing, secreting and/or containing surfaces). In particular, topical application refers to applications that provide little or substantially no systemic delivery of the active compounds in the present compositions. Exemplary mucosal surfaces include the mucosal surfaces of the eyes, mouth (such as the lips, tongue, gums, cheeks, sublingual and roof of the mouth), larynx, esophagus, bronchus, trachea, nasal passages, vagina and rectum/anus.

For oral administration, an active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance. To facilitate drug stability and absorption, peptides of the invention can be released from a capsule after passing through the harsh proteolytic environment of the stomach. Methods for enhancing peptide stability and absorption after oral administration are well known in the art (e.g., Mahato R I. Emerging trends in oral delivery of peptide and protein drugs. Critical Reviews in Therapeutic Drug Carrier Systems. 20:153-214, 2003).

Dosage forms such as lozenges, chewable tablets and chewing gum permit more rapid therapeutic action compared to per-oral dosage forms of the synthetic peptide amide compounds of the invention having significant buccal absorption. Chewing gum formulations are solid, single dose preparations with a base consisting mainly of gum, that are intended to be chewed but not swallowed, and contain one or more compounds of the invention which are released by chewing and are intended to be used for local treatment of pain and inflammation of the mouth or systemic delivery after absorption through the buccal mucosa. See for example, U.S. Pat. No. 6,322,828 to Athanikar and Gubler entitled: Process for manufacturing a pharmaceutical chewing gum.

For nasal administration, the peripherally selective kappa opioid receptor agonists can be formulated as aerosols. The term “aerosol” includes any gas-borne suspended phase of the compounds of the instant invention which is capable of being inhaled into the bronchioles or nasal passages. Specifically, aerosol includes a gas-borne suspension of droplets of the compounds of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159.

The pharmaceutical compositions useful in the practice of the invention can be prepared in a formulation suitable for systemic delivery, such as for instance by intravenous, subcutaneous, intramuscular, intraperitoneal, intranasal, transdermal, intravaginal, intrarectal, intrapulmonary or oral delivery. Alternatively, the pharmaceutical compositions of the invention can be suitably formulated for local delivery, such as, for instance, for topical, or iontophoretic delivery, or for transdermal delivery by a patch coated, diffused or impregnated with the formulation, and local application to the joints, such as by intra-articular injection.

Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous, and thereby formulated for delivery by injection, infusion, or using implantable pumps. For intravenous, subcutaneous, and intramuscular administration, useful formulations of the invention include microcapsule preparations with controlled release properties (R. Pwar et al. Protein and peptide parenteral controlled delivery. Expert Opin Biol Ther. 4(8):1203-12, 2004) or encapsulation in liposomes, with an exemplary form being polyethylene coated liposomes, which are known in the art to have an extended circulation time in the vasculature (e.g. Koppal, T. “Drug delivery technologies are right on target”, Drug Discov. Dev. 6, 49-50, 2003).

For ophthalmic administration, the present invention provides a method of treating glaucoma or ophthalmic pain and inflammation, comprising administering to an eye of a patient in need thereof a therapeutically effective amount of a synthetic peptide amide of the invention. The synthetic peptide amide can be administered topically with an eye-compatible pharmaceutical carrier or non-systemically using a contact lens or intraocular implant that can optionally contain polymers that provide sustained release of the synthetic peptide amide. Such eye-compatible pharmaceutical carriers can include adjuvants, antimicrobial preservatives, surfactants, and viscolyzers etc. It is known in the art that high concentrations of many compounds are irritant to the eye and low concentrations are less irritant; thus the formulation is often designed to include the lowest effective concentrations of active compound, preservative, surfactant, and/or viscolyzer, said viscolyzer preferably having a high surface tension to reduce irritation of the eye while increasing the retention of ophthalmic solutions at the eye surface. Such controlled release of the synthetic peptide amides of the invention can last 6 months to a year for implants, or for shorter periods (3-14 days) for contact lenses. Such implants can be osmotic pumps, biodegradable matrices, or intraocular sustained release devices. Such topical compositions can include a buffered saline solution with or without liposomes.

Aqueous polymeric solutions, aqueous suspensions, ointments, and gels can be used for topical formulations of the synthetic peptide amides of the invention for ocular applications. The aqueous formulations may also contain liposomes for creating a reservoir of the synthetic peptide amide. Certain of these topical formulations are gels which enhance pre-corneal retention without the inconvenience and impairment of vision associated with ointments. The eye-compatible pharmaceutical carrier can also include a biodegradable synthetic polymer. Biodegradable microsphere compositions approved for human use include the polylactides: poly(lactic acid), poly(glycolic acid), and poly(lactic-coglycolic) acid. Additional biodegradable formulations include, but are not limited to: poly(anhydride-co-imide), poly(lactic-glycolic acid), polyethyl-2-cyanoacrylate, polycaprolactone, polyhydroxybutyrate valerate, polyorthoester, and polyethylene-oxide/polybutylene teraphthalate. Intraocular implantation or injection of sustained release compositions that include a synthetic peptide amide of the invention can provide long-term control (ranging from months to years) of intraocular pressure, and thereby avoiding or reducing the need for topical preparations. Useful methods for formulating and dispensing ophthalmic medications are disclosed in U.S. Pat. No. 7,122,579 to Schwartz et al, and in U.S. Pat. No. 7,105,512 to Morizono et al. Methods for formulating ophthalmic medications in contact lenses are disclosed by Gulsen and Chauhan, Ophthalmic drug delivery through contact lenses. Investigative Ophthalmology and Visual Science, (2004) 45:2342-2347.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include sodium chloride for injection, Ringers solution for injection, isotonic dextrose for injection, sterile water for injection, dextrose and lactated Ringers solution for injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfite. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (Tween 80). A sequestering or chelating agent of metal ions such as EDTA can also be incorporated. Pharmaceutical carriers also include ethyl alcohol, polyethylene glycol and propylene glycol for water miscible vehicles and the pH can be adjusted to a physiologically compatible pH by addition of sodium hydroxide, hydrochloric acid, citric acid or lactic acid.

The active ingredient may be administered all at once, or may be divided into a number of smaller doses to be administered at intervals of time, or as a controlled release formulation. The term “controlled release formulation” encompasses formulations that allow the continuous delivery of a synthetic peptide amide of the invention to a subject over a period of time, for example, several days to weeks. Such formulations may be administered subcutaneously or intramuscularly and allow for the continual steady state release of a predetermined amount of compound in the subject over time. The controlled release formulation of synthetic peptide amide may be, for example, a formulation of drug containing polymeric microcapsules, such as those described in U.S. Pat. Nos. 4,677,191 and 4,728,721, incorporated herein by reference. The concentration of the pharmaceutically active compound is adjusted so that administration provides an effective amount to produce a desired effect. The exact dose depends on the age, weight and condition of the patient or animal, as is known in the art. For any particular subject, specific dosage regimens can be adjusted over time according to the individual need and the professional judgment of the person administering or supervising the administration of the formulations. Thus, the concentration ranges set forth herein are exemplary only and are not intended to limit the scope or practice of the claimed invention.

The unit dose parenteral preparations include packaging in an ampoule or prepackaged in a syringe with, or without a needle for delivery. All preparations for parenteral administration are typically sterile, as is practiced in the art. Illustratively, intravenous infusion of a sterile aqueous buffered solution containing an active compound is an effective mode of administration. In another embodiment a sterile aqueous or oily solution or suspension containing the active material can be injected as necessary to produce the desired pharmacological effect.

The pharmaceutical compositions useful in the practice of the invention can be delivered or administered intravenously, transdermally, transmucosally, intranasally, subcutaneously, intramuscularly, orally or topically (such as for example to the eye). The compositions can be administered for prophylaxis or treatment of individuals suffering from, or at risk of a disease or a disorder. Prophylaxis is defined as a measure designed to preserve the health of an individual. For therapeutic applications, a pharmaceutical composition is typically administered to a subject suffering from a disease or disorder, in an amount sufficient to inhibit, prevent, or ameliorate the disease or disorder. An amount adequate to accomplish this is defined as a “therapeutically effective dose.”

The pharmaceutical compositions useful in the practice of the invention can be administered to a mammal for prophylactic or therapeutic purposes in any of the above-described formulations and delivery modes. The mammal can be any mammal, such as a domesticated or feral mammal, or even a wild mammal. The mammal can be any primate, ungulate, canine or feline. For instance, and without limitation, the mammal may be a pet or companion animal, such as a dog or a cat; a high-value mammal such as a thoroughbred horse or a show animal; a farm animal, such as a cow, a goat, a sheep or pig; or a primate such as an ape, gorilla, orangutan, lemur, monkey or chimpanzee. A suitable mammal for prophylaxis or treatment using the pharmaceutical compositions of the invention is a human.

The pharmaceutical compositions useful in the practice of the invention can be administered to a mammal having a disease or condition treatable by activation of the kappa opioid receptor. Alternatively, the pharmaceutical compositions can be administered as prophylactics to a mammal having a risk of contracting or developing a disease or condition preventable by activation of the kappa opioid receptor. Diseases or conditions that can be treated or prevented by administration of the pharmaceutical compositions of the invention include, without limitation, any condition that can be ameliorated by activation of the kappa opioid receptor, including such conditions as pain, inflammation, pruritis, hyponatremia, hypokalemia, congestive heart failure, liver cirrhosis, nephrotic syndrome, hypertension, edema, ileus, tussis and glaucoma.

In a particular embodiment, the pharmaceutical compositions useful in the practice of the invention can be co-administered with or can include one or more other therapeutic compounds or adjuvants, such as but not limited to other opioids, cannabinoids, antidepressants, anticonvulsants, neuroleptics, antihistamines, acetaminophen, corticosteroids, ion channel blocking agents, non-steroidal anti-inflammatory drugs (NSAIDs), and diuretics, many of which are synergistic in effect with the synthetic peptide amides of the invention.

Suitable opioids, include, without limitation, alfentanil, alphaprodine, anileridine, bremazocine, buprenorphine, butorphanol, codeine, conorphone, dextromoramide, dextropropoxyphene, dezocine, diamorphine, dihydrocodeine, dihydromorphine, diphenoxylate, dipipanone, doxpicomine, ethoheptazine, ethylketazocine, ethylmorphine, etorphine, fentanyl, hydrocodone, hydromorphone, ketobemidone, levomethadyl, levorphanol, lofentanil, loperamide, meperidine (pethidine), meptazinol, methadone, morphine, morphine-6-glucuronide, nalbuphine, nalorphine, nicomorphine, oxycodone, oxymorphone, pentazocine, phenazocine, phenoperidine, piritramide, propiram, propoxyphene, remifentanil, sufentanil, tilidate, tonazocine, and tramadol.

One embodiment of the invention is a therapeutic use of a co-formulation and/or therapeutic co-administration of an opioid with substantial agonist activity at the mu opioid receptor, such as morphine, fentanyl, hydromorphone, or oxycodone, together with a synthetic peptide amide of the invention, for the purpose of a mu opioid dose-sparing effect, where the dose of the mu opioid is reduced to minimize common mu opioid side effects, particularly in opioid-naïve patients. Such side effects include constipation, nausea, vomiting, sedation, respiratory depression, pruritis (itching), mental confusion, disorientation and cognitive impairment, urinary retention, biliary spasm, delirium, myoclonic jerks, and seizures. The selection of the reduced mu opioid dose requires expert clinical judgment, and depends on the unique characteristics of the various mu opioids, as well as patient characteristics such as pain intensity, patient age, coexisting disease, current drug regimen and potential drug interactions, prior treatment outcomes, and patient preference (McCaffery, M. and Pasero, C., Pain Clinical Manual, Second Edition, Mosby, 1999).

Cannabinoids suitable for administration with or incorporation into the pharmaceutical compositions of the invention, include any natural cannabinoid, such as for instance, tetrahydrocannabinol (THC), or a THC derivative, or a synthetic cannabinoid, such as, for instance, levonantradol, marinol, nabilone, rimonabant or savitex.

Suitable antidepressants that can be co-administered with or incorporated into the pharmaceutical compositions of the invention, include for example, tricyclic antidepressants such as imipramine, desipramine, trimipramine, protriptyline, norttriptyline, amitriptyline, doxepin, and clomipramine; atypical antidepressants such as amoxapine, maprotiline, trazodone, bupropion, and venlafaxine; serotonin-specific reuptake inhibitors such as fluoxetine, sertraline, paroxetine, citalopram and fluvoxamine; norepinephrine-specific reuptake inhibitors such as reboxetine; or dual-action antidepressants such as nefazodone and mirtazapine.

Suitable neuroleptics that can be co-administered with or incorporated into the pharmaceutical compositions of the invention, include any neuroleptic, for example, a compound with D2 dopamine receptor antagonist activity such as domperidone, metaclopramide, levosulpiride, sulpiride, thiethylperazine, ziprasidone, zotepine, clozapine, chlorpromazine, acetophenazine, carphenazine, chlorprothixene, fluphenazine, loxapine, mesoridazine, molindone, perphenazine, pimozide, piperacetazine, perchlorperazine, thioridazine, thiothixene, trifluoperazine, triflupromazine, pipamperone, amperozide, quietiapine, melperone, remoxipride, haloperidol, rispiridone, olanzepine, sertindole, ziprasidone, amisulpride, prochlorperazine, and thiothixene.

Anticonvulsants such as phenobarbital, phenytoin, primidone, carbamazepine, ethosuximide, lamotrigine, valproic acid, vigabatrin, felbamate, gabapentin, levetiracetam, oxcarbazepine, remacemide, tiagabine, and topiramate can also usefully be incorporated into the pharmaceutical compositions of the invention.

Muscle relaxants such as methocarbamol, orphenadrine, carisoprodol, meprobamate, chlorphenesin carbamate, diazepam, chlordiazepoxide and chlorzoxazone; anti-migraine agents such as sumitriptan, analeptics such as caffeine, methylphenidate, amphetamine and modafinil; antihistamines such as chlorpheniramine, cyproheptadine, promethazine and pyrilamine, as well as corticosteroids such as methylprednisolone, betamethasone, hydrocortisone, prednisolone, cortisone, dexamethasone, prednisone, alclometasone, clobetasol, clocortrolone, desonide, desoximetasone, diflorasone, fluocinolone, fluocinonide, flurandrenolide, fluticasone, floromethalone, halcinonide, halobetasol, loteprednol, mometasone, prednicarbate, and triamcinolone can also be incorporated into the pharmaceutical compositions of the invention.

Ion channel blocking agents such as, for instance, the sodium ion channel blocker, carbamazepine, as commonly used in the treatment of tinnitus, arrhythmia, ischemic stroke and epilepsy can be co-administered with or incorporated into the pharmaceutical compositions of the invention. Alternatively, or in addition, calcium ion channel blockers, such as ziconotide, can also be used, as can antagonists of the ion channel associated with the NMDA receptor, such as ketamine. There is evidence that at least some of these ion channel blockers can potentiate the analgesic effects of the kappa agonist and thereby reduce the dose required for affective pain relief. See for instance, Wang et al., 2000, Pain 84: 271-81.

Suitable NSAIDs, or other non-opioid compounds with anti-inflammatory and/or analgesic activity, that can be co-administered with or incorporated into the pharmaceutical compositions of the invention include, but are not limited to one or more of the following: aminoarylcarboxylic acid derivatives such as etofenamate, meclofenamic acid, mefanamic acid, niflumic acid; arylacetic acid derivatives such as acemetacin, amfenac, cinmetacin, clopirac, diclofenac, fenclofenac, fenclorac, fenclozic acid, fentiazac, glucametacin, isoxepac, lonazolac, metiazinic acid, naproxin, oxametacine, proglumetacin, sulindac, tiaramide and tolmetin; arylbutyric acid derivatives such as butibufen and fenbufen; arylcarboxylic acids such as clidanac, ketorolac and tinoridine. arylpropionic acid derivatives such as bucloxic acid, carprofen, fenoprofen, flunoxaprofen, ibuprofen, ibuproxam, oxaprozin, phenylalkanoic acid derivatives such as flurbiprofen, piketoprofen, pirprofen, pranoprofen, protizinic acid and tiaprofenic acid; pyranocarboxylic acids such as etodolac; pyrazoles such as mepirizole; pyrazolones such as clofezone, feprazone, mofebutazone, oxyphinbutazone, phenylbutazone, phenyl pyrazolidininones, suxibuzone and thiazolinobutazone; salicylic acid derivatives such as aspirin, bromosaligenin, diflusinal, fendosal, glycol salicylate, mesalamine, 1-naphthyl salicylate, magnesium salicylate, olsalazine and salicylamide, salsalate, and sulfasalazine; thiazinecarboxamides such as droxicam, isoxicam and piroxicam others such as ε-acetamidocaproic acid, acetaminophen, s-adenosylmethionine, 3-amino-4-hydroxybutyric acid, amixetrine, bendazac, bucolome, carbazones, cromolyn, difenpiramide, ditazol, hydroxychloroquine, indomethacin, ketoprofen and its active metabolite 6-methoxy-2-naphthylacetic acid; guaiazulene, heterocylic aminoalkyl esters of mycophenolic acid and derivatives, nabumetone, nimesulide, orgotein, oxaceprol, oxazole derivatives, paranyline, pifoxime, 2-substituted-4, 6-di-tertiary-butyl-s-hydroxy-1,3-pyrimidines, proquazone and tenidap, and cox-2 (cyclooxygenase II) inhibitors, such as celecoxib or rofecoxib.

Suitable diuretics that can be co-administered with or incorporated into the pharmaceutical preparations of the invention, include, for example, inhibitors of carbonic anhydrase, such as acetazolamide, dichlorphenamide, and methazolamide; osmotic diuretics, such as glycerin, isosorbide, mannitol, and urea; inhibitors of Na⁺—K⁺-2Cl⁻ symport (loop diuretics or high-ceiling diuretics), such as furosemide, bumetanide, ethacrynic acid, torsemide, axosemide, piretanide, and tripamide; inhibitors of Na⁺—Cl⁻ symport (thiazide and thiazidelike diuretics), such as bendroflumethiazide, chlorothiazide, hydrochlorothiazide, hydroflumethazide, methyclothiazide, polythiazide, trichlormethiazide, chlorthalidone, indapamide, metolazone, and quinethazone; and, in addition, inhibitors of renal epithelial Na⁺ channels, such as amiloride and triamterene, and antagonists of mineralocorticoid receptors (aldosterone antagonists), such as spironolactone, canrenone, potassium canrenoate, and eplerenone, which, together, are also classified as K⁺-sparing diuretics. One embodiment is co-formulation and/or co-administration of a loop or thiazide diuretic together with a synthetic peptide amide of the invention for the purpose of a loop or thiazide diuretic dose-sparing effect, wherein the dose of the loop or thiazide diuretic is reduced to minimize undesired water retention, and prevent or reduce hyponatremia, particularly in the context of congestive heart failure, as well as other medical conditions where decreasing body fluid retention and normalizing sodium balance could be beneficial to a patient in need thereof. See R M Reynolds et al. Disorders of sodium balance Brit. Med. J. 2006; 332:702-705.

The kappa opioid receptor-associated hyponatremia can be any disease or condition where hyponatremia (low sodium condition) is present, e.g., in humans, when the sodium concentration in the plasma falls below 135 mmol/L, an abnormality that can occur in isolation or, more frequently, as a complication of other medical conditions, or as a consequence of using medications that can cause sodium depletion.

A further embodiment is a therapeutic use of a co-formulation and/or co-administration of a potassium-sparing diuretic with a synthetic peptide amide, e.g., a mineralocorticoid receptor antagonist, such as spironolactone or eplerenone, together with a synthetic peptide amide of the invention, for the purpose of enabling a reduced dose of said potassium-sparing diuretic, wherein the dose of said diuretic is reduced to minimize hyperkalemia or metabolic acidosis, e.g., in patients with hepatic cirrhosis.

In particular embodiments, the synthetic peptide amides useful in the practice of the invention exhibit a long lasting duration of action when administered in therapeutically relevant doses in vivo. For instance, in some embodiments, the synthetic peptide amides of the invention when administered to a mammal at a dose of 3 mg/kg of the synthetic peptide amide maintain at least about 50% of maximum efficacy in a kappa opioid receptor-dependent assay at 3 hours post administration. In certain other embodiments, the synthetic peptide amides of the invention when administered to a mammal at a dose of 0.1 mg/kg of the synthetic peptide amide maintain at least about 50% of maximum efficacy in a kappa opioid receptor-dependent assay at 3 hours post administration. The maximum efficacy is operationally defined as the highest level of efficacy determined for the particular kappa opioid receptor-dependent assay for all agonists tested.

In certain embodiments, the synthetic peptide amides useful in the practice of the invention when administered to a mammal at a dose of 0.1 mg/kg maintain at least about 75% of maximum efficacy at 3 hours post administration. In still other embodiments, the synthetic peptide amides of the invention when administered to a mammal at a dose of 0.1 mg/kg maintain at least about 90% of maximum efficacy at 3 hours post administration. In certain other embodiments, the synthetic peptide amides of the invention when administered to a mammal at a dose of 0.1 mg/kg maintain at least about 95% of maximum efficacy at three hours post administration.

The invention further provides a method of treating or preventing a kappa opioid receptor-associated disease or condition in a mammal, wherein the method includes administering to the mammal a composition containing an effective amount of a synthetic peptide amide of the invention. The mammal can be any mammal, such as a domesticated or feral mammal, or even a wild mammal. Alternatively, the mammal can be a primate, an ungulate, a canine or a feline. For instance, and without limitation, the mammal may be a pet or companion animal, such as a high-value mammal such as a thoroughbred or show animal; a farm animal, such as a cow, a goat, a sheep or pig; or a primate such as an ape or monkey. In one particular aspect, the mammal is a human.

The effective amount can be determined according to routine methods by one of ordinary skill in the art. For instance, an effective amount can be determined as a dosage unit sufficient to prevent or to treat a kappa receptor-associated disease or condition in the mammal. Alternatively, the effective amount may be determined as an amount sufficient to approximate the EC₅₀ concentration or an amount sufficient to approximate two or three times or up to about five or even about ten times the EC₅₀ concentration in a therapeutically relevant body fluid of the mammal, for instance, where the body fluid is in direct apposition to a target tissue, such as the synovial fluid of an inflamed joint in a patient suffering from rheumatoid arthritis.

In one embodiment the synthetic peptide amide useful in the practice of the invention is a pharmaceutical composition that includes an effective amount of the synthetic peptide amide of the invention and a pharmaceutically acceptable excipient or carrier. In one aspect, the pharmaceutical composition includes a synthetic peptide amide of the invention in an amount effective to treat or prevent a kappa opioid receptor-associated condition in a mammal, such as a human. In another aspect the kappa opioid receptor-associated condition is pain, inflammation, pruritis, edema, ileus, tussis or glaucoma.

In one embodiment the pharmaceutical composition useful in the practice of the invention further includes one or more of the following compounds: an opioid, a cannabinoid, an antidepressant, an anticonvulsant, a neuroleptic, a corticosteroid, an ion channel blocking agent or a non-steroidal anti-inflammatory drug (NSAID).

Pharmaceutical compositions of a synthetic peptide amide useful in the practice of the invention and a pharmaceutically acceptable vehicle or carrier can be used to treat or prevent one or more of a variety of kappa opioid receptor-associated diseases, disorders or conditions.

The kappa opioid receptor-associated disease, disorders or condition preventable or treatable with the synthetic peptide amides of the invention can be any kappa opioid receptor-associated condition, including but not limited to acute or chronic pain, inflammation, pruritis, hyponatremia, edema, ileus, tussis and glaucoma. For instance, the kappa opioid receptor-associated pain can be neuropathic pain, somatic pain, visceral pain or cutaneous pain. Some diseases, disorders, or conditions are associated with more than one form of pain, e.g., postoperative pain can have any or all of neuropathic, somatic, visceral, and cutaneous pain components, depending upon the type and extent of surgical procedure employed.

The kappa opioid receptor-associated inflammation can be any inflammatory disease or condition including, but not limited to sinusitis, rheumatoid arthritis tenosynovitis, bursitis, tendonitis, lateral epicondylitis, adhesive capsulitis, osteomyelitis, osteoarthritic inflammation, inflammatory bowel disease (IBD), irritable bowel syndrome (IBS), ocular inflammation, otitic inflammation or autoimmune inflammation.

The kappa opioid receptor-associated pruritis can be any pruritic disease or condition such as, for instance, ocular pruritis, e.g., associated with conjunctivitis, otitic pruritis, pruritis associated with end-stage renal disease, where many patients are receiving kidney dialysis, and other forms of cholestasis, including primary biliary cirrhosis, intrahepatic cholestasis of pregnancy, chronic cholestatic liver disease, uremia, malignant cholestasis, jaundice, as well as dermatological conditions such as eczema (dermatitis), including atopic or contact dermatitis, psoriasis, polycythemia vera, lichen planus, lichen simplex chronicus, pediculosis (lice), thyrotoxicosis, tinea pedis, urticaria, scabies, vaginitis, anal pruritis associated with hemorrhoids and, as well as insect bite pruritis and drug-induced pruritis, such as mu opioid-induced pruritis.

The kappa opioid receptor-associated edema can be any edematous disease or condition such as, for instance, edema due to congestive heart disease or to a syndrome of inappropriate antidiuretic hormone (ADH) secretion.

Kappa opioid receptor-associated ileus can be any ileus disease or condition including, but not limited to, post-operative ileus and opioid-induced bowel dysfunction.

Kappa opioid receptor-associated neuropathic pain can be any neuropathic pain, such as, for instance, trigeminal neuralgia, diabetic pain, viral pain such as herpes zoster-associated pain, chemotherapy-induced pain, nerve-encroaching metastatic cancer pain, neuropathic pain associated with traumatic injury and surgical procedures, as well as variants of headache pain that are thought to have a neuropathic component, e.g., migraine.

Kappa opioid-associated pain also includes ocular pain, such as that following photo-refractive keratectomy (PRK), ocular laceration, orbital floor fracture, chemical burns, corneal abrasion or irritation, or pain associated with conjunctivitis, corneal ulcers, scleritis, episcleritis, sclerokeratitis, herpes zoster ophthalmicus, interstitisal keratitis, acute iritis, keratoconjunctivitis sicca, orbital cellulites, orbital pseudotumor, pemphigus, trachoma or uveitis.

Kappa opioid-associated pain also includes throat pain, particularly associated with inflammatory conditions, such as allergic rhinitis, acute bronchitis, the common cold, contact ulcers, herpes simplex viral lesions, infectious mononucleosis, influenza, laryngeal cancer, acute laryngitis, acute necrotizing ulcerative gingivitis, peritonsillar abscess, pharyngeal burns, pharyngitis, reflus laryngopharyngitis, acute sinusitis, and tonsillitis.

In addition, kappa opioid receptor-associated pain can be arthritic pain, kidney-stone, urinary tract stone, gallstone, and bile duct stone pain, dysmenorrhea, uterine cramping, endometriosis, mastitis, dyspepsia, post-surgical pain (such as, for instance, from appendectomy, open colorectal surgery, hernia repair, prostatectomy, colonic resection, gastrectomy, splenectomy, colectomy, colostomy, pelvic laparoscopy, tubal ligation, hysterectomy, vasectomy or cholecystectomy), post medical procedure pain (such as, for instance, after colonoscopy, cystoscopy, hysteroscopy or cervical or endometrial biopsy), otitic pain, breakthrough cancer pain, and pain associated with a GI disorder such as IBD or IBS or other inflammatory conditions, particularly of the viscera (e.g., gastro-esophageal reflux disease, pancreatitis, acute polynephritis, ulcerative colitis, acute pyelo-nephritis, cholecystitis, cirrhosis, hepatic abscess, hepatitis, duodenal or gastric ulcer, esophagitis, gastritis, gastroenteritis, colitis, diverticulitis, intestinal obstruction, ovarian cyst, pelvic inflammatory disease, perforated ulcer, peritonitis, prostatitis, interstitial cystitis), or exposure to toxic agents, such as insect toxins, or inflammation due to the effects of drugs such as salicylates or NSAIDs.

The present invention provides a method of treating or preventing a kappa opioid receptor-associated disease or condition in a mammal, such as a human, wherein the method includes administering to the mammal a composition comprising an effective amount of a synthetic peptide amide of the invention. In another embodiment the kappa opioid receptor-associated condition is pain, inflammation (such as rheumatoid arthritic inflammation, osteoarthritic inflammation, IBD inflammation, IBS inflammation, ocular inflammation, otitic inflammation or autoimmune inflammation), pruritis (such as atopic dermatitis, kidney-dialysis-associated pruritis, ocular pruritis, otitic pruritis, insect bite pruritis, or opioid-induced pruritis), edema, ileus, tussis or glaucoma. In one aspect, the pain is a neuropathic pain (such as trigeminal neuralgia, migraine, diabetic pain, viral pain, chemotherapy-induced pain or metastatic cancer pain), a somatic pain, a visceral pain or a cutaneous pain. In another aspect the pain is arthritic pain, kidney-stone pain, uterine cramping, dysmenorrhea, endometriosis, dyspepsia, post-surgical pain, post medical procedure pain, ocular pain, otitic pain, breakthrough cancer pain or pain associated with a GI disorder, such as IBD or IBS. In another aspect the pain is pain associated with surgery, wherein the surgery is pelvic laparoscopy, tubal ligation, hysterectomy and cholecystecomy. Alternatively, the pain can be pain associated with a medical procedure, such as for instance, colonoscopy, cystoscopy, hysteroscopy or endometrial biopsy. In a specific aspect, the atopic dermatitis can be psoriasis, eczema or contact dermatitis. In another specific aspect, the ileus is post-operative ileus or opioid-induced bowel dysfunction.

Kappa opioid receptor-associated pain includes hyperalgesia, which is believed to be caused by changes in the milieu of the peripheral sensory terminal occur secondary to local tissue damage. Tissue damage (e.g., abrasions, burns) and inflammation can produce significant increases in the excitability of polymodal nociceptors (C fibers) and high threshold mechanoreceptors (Handwerker et al. (1991) Proceeding of the VIth World Congress on Pain, Bond et al., eds., Elsevier Science Publishers BV, pp. 59-70; Schaible et al. (1993) Pain 55:5-54). This increased excitability and exaggerated responses of sensory afferents is believed to underlie hyperalgesia, where the pain response is the result of an exaggerated response to a stimulus. The importance of the hyperalgesic state in the post-injury pain state has been repeatedly demonstrated and appears to account for a major proportion of the post-injury/inflammatory pain state. See for example, Woold et al. (1993) Anesthesia and Analgesia 77:362-79; Dubner et al. (1994) In, Textbook of Pain, Melzack et al., eds., Churchill-Livingstone, London, pp. 225-242.

In another embodiment the kappa opioid receptor-associated condition is pain, inflammation (such as rheumatoid arthritic inflammation, osteoarthritic inflammation, IBD inflammation, IBS inflammation, ocular inflammation, otitic inflammation or autoimmune inflammation), pruritis (such as atopic dermatitis, kidney-dialysis-associated pruritis, ocular pruritis, otitic pruritis, insect bite pruritis, or opioid-induced pruritis), edema, ileus, tussis or glaucoma. In one aspect, the pain is a neuropathic pain (such as trigeminal neuralgia, migraine, diabetic pain, viral pain, chemotherapy-induced pain or metastatic cancer pain), a somatic pain, a visceral pain or a cutaneous pain. In another aspect the pain is arthritic pain, kidney-stone pain, uterine cramping, dysmenorrhea, endometriosis, dyspepsia, post-surgical pain, post medical procedure pain, ocular pain, otitic pain, breakthrough cancer pain or pain associated with a GI disorder, such as IBD or IBS. In another aspect the pain is pain associated with surgery, wherein the surgery is pelvic laparoscopy, tubal ligation, hysterectomy and cholecystecomy. Alternatively, the pain can be pain associated with a medical procedure, such as for instance, colonoscopy, cystoscopy, hysteroscopy or endometrial biopsy. In a specific aspect, the atopic dermatitis can be psoriasis, eczema or contact dermatitis. In another specific aspect, the ileus is post-operative ileus or opioid-induced bowel dysfunction.

In another embodiment the kappa opioid receptor-associated condition is a kappa opioid receptor-associated condition preventable or treatable by sodium and potassium-sparing diuresis, also known as aquaresis. An example of such kappa opioid receptor-associated conditions preventable or treatable by administering a synthetic peptide amide of the invention includes edema. The edema may be due to any of a variety of diseases or conditions, such as congestive heart disease or syndrome of inappropriate ADH secretion.

In another embodiment the kappa opioid receptor-associated condition is hyponatremia or other edematous disease. The kappa opioid receptor-associated hyponatremia or edema can be any hyponatremic or edematous disease or condition such as, for instance, hyponatremia and edema associated with congestive heart failure or to a syndrome of inappropriate antidiuretic hormone (ADH) secretion, or hyponatremia that is associated with intensive diuretic therapy with thiazides and/or loop diuretics. The synthetic peptide amides of the invention exhibit a significant sodium-sparing and potassium-sparing aquaretic effect, which is beneficial in the treatment of edema-forming pathological conditions associated with hyponatremia and/or hypokalemia. Accordingly, the synthetic peptide amides of the invention also have utility in methods of treating or preventing hyponatremia-related conditions, examples of which are provided below. Hyponatremia-related conditions can be categorized according to volume status as hypervolemic, euvolemic, or hypovolemic.

Hypervolemic hyponatremia is usually caused by an increase in total body water level as may be observed in cases of congestive heart failure, nephrotic syndrome and hepatic cirrhosis.

Euvolemic hyponatremia is often found in the syndrome of inappropriate antidiuretic hormone (ADH) secretion and may also be associated with pneumonia, small-cell lung cancer, polydipsia, cases of head injury, and organic causes (e.g., use of certain drugs, such as haloperidol) or a psychogenic cause.

Hypovolemic hyponatremia is due to a relative decrease in total body sodium level and may be associated with, for instance and without limitation, diuretic use, cases of interstitial nephritis or excessive sweating.

These forms of hyponatremia can be further classified according to the concentration of sodium in the urine (i.e., whether the concentration is greater than or less than thirty millimoles per liter. See: R M Reynolds et al. Disorders of sodium balance, Brit. Med. J. 2006; 332:702-705.

The kappa opioid receptor-associated hyponatremia can be any disease or condition where hyponatremia (low sodium condition) is present, e.g., in humans, when the sodium concentration in the plasma falls below 135 mmol/L, an abnormality that can occur in isolation or, more frequently, as a complication of other medical conditions, or as a consequence of using medications that can cause sodium depletion.

In addition to these conditions, numerous other conditions are associated with hyponatremia including, without limitation: neoplastic causes of excess ADH secretion, including carcinomas of lung, duodenum, pancreas, ovary, bladder, and ureter, thymoma, mesothelioma, bronchial adenoma, carcinoid, gangliocytoma and Ewing's sarcoma; infections such as: pneumonia (bacterial or viral), abscesses (lung or brain), cavitation (aspergillosis), tuberculosis (lung or brain), meningitis (bacterial or viral), encephalitis and AIDS; vascular causes such as: cerebrovascular occlusions or hemorrhage and cavernous sinus thrombosis; neurologic causes such as: Guillan-Barre syndrome, multiple sclerosis, delirium tremens, amyotrophic lateral sclerosis, hydrocephalus, psychosis, peripheral neuropathy, head trauma (closed and penetrating), CNS tumors or infections and CNS insults affecting hypothalamic osmoreceptors; congenital malformations including: agenesis of corpus callosum, cleftlip/palate and other midline defects; metabolic causes such as: acute intermittent porphyria, asthma, pneurothorax and positive-pressure respiration; drugs such as: thiazide diuretics, acetaminophen, barbiturates, cholinergic agents, estrogen, oral hypoglycemic agents, vasopressin or desmopressin, high-dose oxytocin, chlorpropamide, vincristine, carbamezepine, nicotine, phenothiazines, cyclophosphamide, tricyclic antidepressants, monoamine oxidase inhibitors and serotonin reuptake inhibitors; administration of excess hypotonic fluids, e.g., during hospitalization, surgery, or during or after athletic events (i.e., exercise-associated hyponatremia), as well as use of low-sodium nutritional supplements in elderly individuals. See for example, Harrison's Principles of Internal Medicine, 16th Ed. (2005), p. 2102.

Other conditions associated with hyponatremia include renal failure, nephrotic syndrome (membranous nephropathy and minimal change disease), cachexia, malnutrition, rhabdomyolysis, surgical procedures, elective cardiac catheterization, blood loss, as well as hypercalcemia, hypokalemia, and hyperglycemia with consequent glycosuria leading to osmotic diuresis.

The invention also provides a method of treating or preventing a neuro-degenerative disease or condition in a mammal, such as a human, wherein the method includes administering to the mammal a composition that includes an effective amount of a synthetic peptide amide as described above. The neurodegenerative disease or condition can be any neurodegenerative disease or condition, such as for instance, ischemia, anoxia, stroke, brain injury, spinal cord injury or reperfusion injury. Alternatively, the neurodegenerative disease or condition can be a neurodegenerative disease of the eye. Particular neurodegenerative diseases of the eye treatable or preventable by the method of the invention include glaucoma, macular degeneration, retinal ischemic disease and diabetic neuropathy.

In certain embodiments the invention provides methods of prevention or treatment of certain neuronal diseases and conditions, such as diseases and conditions having a neurodegenerative component. Synthetic peptide amides of the invention can be administered in an amount effective to protect neuronal cells against the effects of pathology or injury that would lead to neurodegeneration and/or neuronal cell death of the untreated cells. For example, several diseases or conditions of the eye that have a neurodegenerative component can be prevented or treated by administration of an effective amount of the synthetic peptide amides of the invention. Such diseases and conditions of the eye include glaucoma, macular degeneration, retinal ischemic disease and diabetic neuropathy. Progression of these diseases and conditions is believed to involve neurodegeneration or neuronal cell death, for example by programmed cell death (apoptosis) in which the neuronal cells are committed to a pathway that without intervention would lead to cell death. It has been found that development or progression of these diseases and conditions can be prevented, or at least slowed, by treatment with kappa opioid receptor agonists. This improved outcome is believed to be due to neuroprotection by the kappa opioid receptor agonists. See for instance, Kaushik et al. “Neuroprotection in Glaucoma” (2003) J. Postgraduate Medicine vol. 49 (1): pp. 90-95.

In the case of glaucoma it is believed that prophylaxis and treatment by administration of kappa opioid receptor agonists is mediated by at least two distinct activities induced by activation of the kappa opioid receptor: neuroprotection and reduction of intraocular pressure (TOP). While not wishing to be bound by theory, it is believed that neuroprotection is due, at least in part, to induction of atrial natriuretic peptide (ANP) in the eye, leading to protection against oxidative damage and other insults.

Abnormally high intraocular pressure is also believed to be a factor leading to the development of glaucoma. Elevated intraocular pressure can also be prevented or treated by administration of kappa opioid receptor agonists by three separate activities triggered by activation of the receptor: reduction in secretion of aqueous humor, increased outflow of aqueous humor and aquaresis (sodium- and potassium-sparing diuresis, resulting in loss of water).

The invention also provides a method of treating or preventing a kappa-receptor-associated disease or condition of the eye of a mammal, such as high intraocular pressure (IOP). The method includes administering to the mammal a composition that includes an effective amount of a synthetic peptide amide as described above. In one aspect of the invention, the synthetic peptide amide is administered topically. In another aspect, the synthetic peptide amide is administered as an implant.

In other embodiments the invention provides methods of prevention or treatment of certain cardiovascular diseases and conditions having a cellular degenerative component. Synthetic peptide amides of the invention can be administered in an amount effective to protect myocardial cells against the effects of pathology or injury that would lead to degeneration and/or cell death of the untreated cells. For example, several cardiovascular diseases or conditions can be prevented or treated by administration of an effective amount of the synthetic peptide amides of the invention. Such cardiovascular diseases and conditions include, without limitation, coronary heart disease, ischemia, cardiac infarct, reperfusion injury and arrhythmia. See for example, Wu et al. “Cardioprotection of Preconditioning by Metabolic Inhibition in the Rat Ventricular Myocyte—Involvement of kappa Opioid Receptor” (1999) Circulation Res vol. 84: pp. 1388-1395. See also Yu et al. “Anti-Arrythmic Effect of kappa Opioid Receptor Stimulation in the Perfused Rat Heart: Involvement of a cAMP-Dependent Pathway” (1999) J Mol Cell Cardiol. vol. 31(10): pp. 1809-1819.

Diseases and conditions of other tissues and organs that can be prevented or treated by administration of an effective amount of the synthetic peptide amides of the invention include, but are not limited to ischemia, anoxia, stroke, brain or spinal cord injury and reperfusion injury.

Another form of kappa opioid receptor-associated pain treatable or preventable with the synthetic peptide amides of the invention is hyperalgesia. In one embodiment, the method includes administering an effective amount of a synthetic peptide amide of the invention to a mammal suffering from or at risk of developing hyperalgesia to prevent, ameliorate or completely alleviate the hyperalgesia.

The synthetic peptide amides of the invention can be administered by methods disclosed herein for the treatment or prevention of any hyperalgesic condition, such as, but without limitation, a hyperalgesic condition associated with allergic dermatitis, contact dermatitis, skin ulcers, inflammation, rashes, fungal irritation and hyperalgesic conditions associated with infectious agents, burns, abrasions, bruises, contusions, frostbite, rashes, acne, insect bites/stings, skin ulcers, mucositis, gingivitis, bronchitis, laryngitis, sore throat, shingles, fungal irritation, fever blisters, boils, Plantar's warts, surgical procedures or vaginal lesions. For instance, the synthetic peptide amides of the invention can be administered topically to a mucosal surface, such as the mouth, esophagus or larynx, or to the bronchial or nasal passages. Alternatively, the synthetic peptide amides of the invention can be administered topically to the vagina or rectum/anus.

Moreover, the synthetic peptide amides of the invention can be administered by methods disclosed herein for the treatment or prevention of any hyperalgesic condition associated with burns, abrasions, bruises, abrasions (such as corneal abrasions), contusions, frostbite, rashes, acne, insect bites/stings, skin ulcers (for instance, diabetic ulcers or a decubitus ulcers), mucositis, inflammation, gingivitis, bronchitis, laryngitis, sore throat, shingles, fungal irritation (such as athlete's foot or jock itch), fever blisters, boils, Plantar's warts or vaginal lesions (such as vaginal lesions associated with mycosis or sexually transmitted diseases). Methods contemplated for administration of the synthetic peptide amides of the invention for the treatment or prevention of hyperalgesia include those wherein the compound is topically applied to a surface in the eyes, mouth, larynx, esophagus, bronchial, nasal passages, vagina or rectum/anus.

Hyperalgesic conditions associated with post-surgery recovery can also be addressed by administration of the synthetic peptide amides of the invention. The hyperalgesic conditions associated with post-surgery recovery can be any hyperalgesic conditions associated with post-surgery recovery, such as for instance, radial keratectomy, tooth extraction, lumpectomy, episiotomy, laparoscopy and arthroscopy.

Hyperalgesic conditions associated with inflammation are also addressable by administration of the synthetic peptide amides of the invention. Periodontal inflammation, orthodontic inflammation, inflammatory conjunctivitis, hemorrhoids and venereal inflammations can be treated or prevented by topical or local administration of the synthetic peptide amides of the invention.

The invention also provides a method of inducing diuresis in a mammal in need thereof. The method includes administering to the mammal a composition comprising an effective amount of a synthetic peptide amide of the invention as described above.

The invention provides a method of prevention, inhibition or treatment of a patient suffering from uremic pruritus: the method includes administering an effective amount of a kappa opioid receptor agonist to a patient undergoing a dialysis regimen on one or more days in which the dialysis occurs. The weekly schedule for administration of the kappa opioid receptor agonist may be on one dialysis day, or two or three of the dialysis days. In one embodiment of the kappa opioid receptor agonist regimen, the kappa opioid receptor agonist is administered three times per week for at least one week.

In another embodiment of the kappa opioid receptor agonist treatment regimen, at least one administration of the kappa opioid receptor agonist is administered within one hour after a dialysis treatment. In another embodiment at least one administration of the kappa opioid receptor agonist is within fifteen minutes after a dialysis procedure. In one embodiment the kappa opioid receptor agonist may be administered by intravenous injection, e.g. in an IV bolus injection.

In another embodiment the effective amount of the kappa opioid receptor agonist can be estimated from a patient's dry weight. For instance, the effective amount of the kappa opioid receptor agonist administered may be from about 0.1 μg/kg of patient's dry weight to about 5.0 μg/kg of patient's dry weight. In another embodiment, the effective amount of the kappa opioid receptor agonist administered may be from about 0.5 μg/kg of patient's dry weight to about 2.5 μg/kg of patient's dry weight. In another embodiment, the effective amount of the kappa opioid receptor agonist administered is about 1.0 μg/kg of patient's dry weight. In another embodiment, the effective amount of the kappa opioid receptor agonist administered is about 2.5 μg/kg of patient's dry weight.

The present invention also provides a method of reducing an adverse symptom associated with dialysis in a patient undergoing dialysis, the method includes administering an effective amount of a kappa opioid receptor agonist to the patient. Adverse symptoms that may be prevented, inhibited or treated by the methods of the present invention include uremic pruritus, sleep disruption, and mood alteration. The sleep disruption may be pruritus-associated sleep disruption, wherein the patient is roused from sleep by itching. In one embodiment, the mood alteration that may be inhibited or treated by the methods of the present invention is depression.

In one embodiment, the invention further provides a method of prevention, inhibition or treatment of a patient suffering from uremic pruritus: the method includes administering an effective amount of a peripherally-restricted kappa opioid receptor agonist to a patient undergoing a dialysis regimen on one or more days in which the dialysis occurs.

In another embodiment, the invention provides a method of prevention, inhibition or treatment of a patient suffering from uremic pruritus: the method includes administering an effective amount of CR845 to a patient undergoing a dialysis regimen on one or more days in which the dialysis occurs.

### Definitions

The term: kappa opioid receptor—as used herein means the class of opioid receptors designated “Kappa” or “κ” of the four classes of receptors (Mu, Kappa, Delta and ORL1, a receptor type identified by genetic homology) that bind opiates, found in the brain, spinal column and peripheral neurons.

The term: kappa opioid receptor agonist—means a compound that binds and activates a kappa opioid receptor. CR845 is an example of a kappa opioid receptor agonist. Other kappa opioid receptor agonists include asimadoline, TRK-820, Remitch® and nalbuphine. Asimadoline, a selective, non-peptidic kappa-opioid receptor agonists is also useful in methods according to the present invention Asimadoline has the diarylacetamide structure shown below:

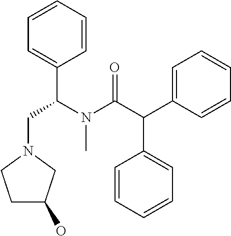 [SMILES: CN(C(=O)C(c1ccccc1)c1ccccc1)[C@H](CN1CC[C@H](O)C1)c1ccccc1]

(N-[(1S)-2-[(3S)-3-hydroxypyrrolidin-1-yl]-1-phenylethyl]-N-methyl-2,2-diphenylacetamide).

Nalfurafine (also known as AC-820, TRK-820) is a kappa opioid receptor agonist marketed for orally delivered opioid treatment for uremic pruritus in hemodialysis patients. Nalfurafine is another kappa opioid receptor agonist useful according to the present invention. Nalfurafine is (2E)-N-[(5α,6β)-17-(cyclopropylmethyl)-3,14-dihydroxy-4,5-epoxymorphinan-6-yl]-3-(3-furyl)-N-methylacrylamide, and has the following chemical structure:

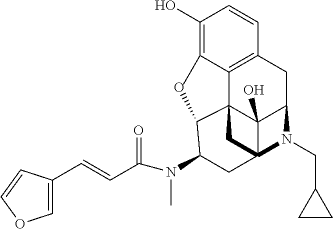

The term: peripheral kappa opioid receptor agonist—means a peripherally-restricted compound (e.g. CR845 or other member of the class of D-amino acid peptide amides disclosed in U.S. Pat. Nos. 7,402,564; 7,727,963; 8,217,007; 8,951,970; and 7,713,937) that binds and activates a kappa opioid receptor, i.e. a kappa opioid receptor agonist that shows little or no CNS effects.

The term: effective amount—means an amount that is sufficient to provide the stated effect.

The term: patient's dry weight—means the weight of the patient when in the normal hydration state, i.e. not suffering from excess fluid retention. The patient's dry weight is usually estimated by a physician and is intended to be similar to the patient's weight with normal kidney function after urinating. It is the lowest weight that the patient can safely reach after dialysis without developing symptoms of low blood pressure, such as cramping, which can occur when too much fluid is removed.

The term: dose means the amount by weight of therapeutic compound (e.g. CR845) administered.

The term: (m/kg) dry weight—means the amount in micrograms per kilogram of patient's dry weight as defined above.

The term: dialysis interchangeably referred to herein as haemodialysis—means the artificial filtration process commonly provided for patients suffering from renal dysfunction.

The term: regimen/schedule—means the dose and timing of administration to the patient of a therapeutic compound.

The term: I.V. bolus injection—means injection of discrete amount to achieve an effective concentration, e.g. by I.V. push.

The term: adverse symptom—means an unfavorable departure from normal signs, e.g. nausea, dizziness, dry mouth, chills and shivering, etc.

One example of the kappa opioid receptor agonist synthetic peptide amides is CR845:

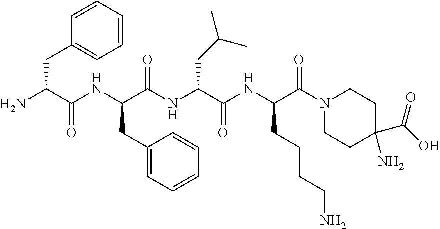 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

CR845: D-Phe-D-Phe-D-Leu-D-Lys-[ω(4-aminopiperidine-4-carboxylic acid)]-OH

Asimadoline is a kappa opioid receptor agonist that acts that has been investigated as a possible treatment for irritable bowel syndrome (IBS). See Szarka et al., Clin. Gastroenterol. Hepatol. 200S7 November; 5(11):1268-1275.

Nalbuphine—(Nubain®) is a dual mu and kappa opioid receptor agonist that has been tested in an extended release formulation in hemodialysis patients as a potential therapy for pruritus. See Hawi et al. (2015) BMC Nephrology 16:47.

Remitch® (Nalfurafine HCl) from Toray Industries is a kappa opioid receptor agonist that has also been investigated in an oral formulation as a possible treatment for pruritus in hemodialysis patients. See Kumagai et al. Am. J. Nephrol. 2012; 36(2):175-183.

Surprisingly, the method of prevention, inhibition or treatment of a patient suffering from uremic pruritus of the invention, that includes administering an effective amount of CR845 to a patient undergoing a dialysis regimen on one or more days in which the dialysis occurs, results in an unexpectedly sustained, long lasting reduction in pruritus in the patient.

Without wishing to be bound by theory, it is believed that this sustained and long lasting reduction in pruritus is due to the unique pharmacokinetics of CR845 and related D-amino acid peptide amides, which are processed by the kidney and excreted in the urine in normal individuals. Thus, dialysis patients suffering kidney dysfunction do not excrete CR845, which remains in the blood and is only cleared by the dialysis procedure. After administration of an effective amount of CR845 within 15 minutes, 30 minutes or up to one to two hours after dialysis, patients experience surprisingly low levels of pruritus.

Remarkably, the reduction in pruritus as judged by the patient on a visual analog scale (VAS), can be greater than 50%, in many cases greater than 75%, in a significant number of cases greater than 90%, and occasionally reach as much as a 98% reduction in patient assessed itch on the visual analog scale. Before the present invention, such high levels of relief from itching were not achievable.

Furthermore, administration of CR845 provides relief from other dialysis-associated adverse symptoms in addition to pruritus, such as but not limited to sleep disorders, including sleep disruption, moodiness and depression.

### Clinical Trials

A multi-center, randomized, double-blind, placebo-controlled, study (CR845-CLIN2005) was conducted in two parts to evaluate the safety, Pharmacokinetics (PK), and efficacy of repeated doses of CR845 administered as intravenous (I.V.) boluses to haemodialysis patients.

Part A was conducted in a clinical research unit with capabilities of performing haemodialysis and keeping patients overnight. Patients in Part A received one of three doses of CR845 or placebo in a dose escalation following a sequential group design.

Part B was conducted in outpatient dialysis units where patients were normally dialyzed. Patients in Part B received CR845 or placebo in a parallel group design.

Study Design Part A

One of three doses of CR845 or matched placebo were administered as an IV bolus once immediately after each dialysis session for one week. Each dose cohort was comprised of twenty-four patients (6 CR845 and 2 placebo for each of three dose levels: See below).

Part A consisted of a Screening visit, Treatment period, and Follow-up visit (approximately 1 week after the last dose). Vitals signs, physical examinations, 12-lead ECG, clinical laboratory tests and urine output in patients who were not anuric (i.e., at least 1 cup/day of urine output by history) were monitored periodically and adverse events (AEs) and concomitant medications were continuously recorded during the study.

Study Design Part B

One dose of CR845 or placebo was administered once immediately after each dialysis session for a period of 2 weeks in 65 patients (blindly randomized to approximately 50% of the patients in the CR845 group and approximately 50% patients in the placebo group).

Part B consists of a Screening visit, a one-week Run-in Period (Baseline), a Treatment Period of 2 weeks and a Follow-up Visit (approximately 1 week after the last dose). Patients report Daytime and Nighttime Worst Itching VAS scores daily during the entire Treatment Period. In addition, during selected study visits, patients completed their additional patient reported outcomes (PROs) (i.e., Itch MOS, Patient Self-Categorization of Pruritus Disease Severity and Skindex-10). Vital signs, physical examinations, 12-lead ECG, and clinical laboratory tests were monitored periodically and AEs and concomitant medications were recorded continuously during the study.

Endpoints

Safety Endpoint (Parts A and B)

The safety endpoint is the overall safety and tolerability of CR845 as assessed by the frequency and severity of AEs by treatment group, physical examination, vital signs, 12-lead ECG, and clinical safety laboratory evaluations. All patients receiving any study drug were included in the safety analysis.

Pharmacokinetic Endpoints (Part A): The PK profile of CR845 with dosing after each dialysis session over a 1 week treatment period (three times per week) was recorded.

Primary Efficacy Endpoints (Part B): The change from baseline to the average of Week 2 worst itching (daytime and night time) visual analog scale (VAS). The mean of the last 8 VAS scores from Week 2 was used for comparison.

Secondary Efficacy Endpoints (Part B): The change from baseline to Day 15 in itch-related quality of life as assessed by the total Skindex-10 scores; Change from baseline to Day 15 in itch-related sleep disturbance based on the Itch MOS sleep problems index II (SLP-9); and Percentage of patients with Patient Assessed Self-categorization of Pruritus Disease Severity with a reduction in category from “B” or “C” from Screening to Day 15.

Duration of Treatment

In Part A, the duration of treatment for each individual patient was one week for a total of three doses of study drug. The overall study duration for each individual patient in Part A was about 5.5 weeks.

In Part B, the duration of treatment for each individual patient was two weeks for a total of six doses of study drug. The overall study duration for each individual patient in Part B was up to 6.5 weeks.

Study Procedures

Part A

Screening

To be included in the trial, the patient had to have completed screening within 21 days prior to the beginning of the Treatment Period. After written informed consent was obtained, the following procedures and assessments were run:

Medical and medication history were recorded: Any adverse events (AEs) that occurred during the Screening Period following signing of the informed consent form (ICF) were recorded as Medical History. Height, weight, vital signs, including BP, HR, RR, and temperature and physical examination results, including examination of the heart, lungs, abdomen, extremities, neurological system, and vascular system were recorded. A blood sample was taken for central laboratory pregnancy test for women of child-bearing potential or FSH assay for post menopausal women and a drug test was performed. Patient Questionnaires: VAS Worst Itching Day and Night for prior 24 hour period was completed in the clinic, within 6 hours prior to starting dialysis whenever possible.

Treatment Period

Part A—Day −1

Admission to clinic: Patients were admitted 24 hours prior to the Day 1 dialysis. This day, Day −1 is the day prior to the patient's usual dialysis day. The 24-hour urine collection was begun and fluid intake was recorded. The urine collection was planned so that the collection was completed prior to starting dialysis on the following day. The total 24-hour urine volume was recorded and an aliquot submitted to the laboratory for urine sodium and creatinine measurement. Fluid intake (oral and I.V.) was recorded during the same 24-hour period as urine volume. Patient questionnaires: VAS Worst Itching Day and Night for prior 24 hour period were completed in the clinic. The erythropoiesis stimulating agent was administered to the patient after fasting overnight in the clinical research unit. Concomitant medications were recorded.

Dialysis

The dialysis prescription was kept constant throughout the study, unless absolutely necessary for patient safety. All procedural data (start and stop times, net ultrafiltration, access changes, dialysis bath sodium concentration, ESA usage) were recorded.

Part A

Study drug was administered as an I.V. bolus via I.V. push into dialysis venous line (e.g., into the venous port) within 15 minutes following the end of dialysis (i.e., return of blood to the patient) on scheduled drug administration days. Following the bolus, the venous line was flushed with at least 10 mL of normal saline. The patient's estimated dry weight (i.e., the target post-dialysis weight, as determined by the patient's nephrologist or dialysis unit) was used to calculate the dose of the study drug.

Individual doses of CR845 or placebo were prepared by an unblinded pharmacist (or qualified staff) from one vial in a sterile environment (e.g. a sterile hood) by withdrawal of a patient-specific volume of CR845 or placebo, up to 24 hours prior to administration. Doses for patients in Group 1 (0.5 μg/kg) were prepared from CR845 0.05 mg/mL; doses for patients in Group 2 (1 μg/kg) were prepared from CR845 0.10 mg/mL; and doses for patients in Group 3 (2.5 μg/kg) were prepared from CR845 0.25 mg/mL.

Placebo doses for each group were prepared from the placebo vials. For each dose, an appropriate volume of CR845 or placebo solution (based upon the patient's estimated dry weight) was drawn up from 1 vial using a sterile syringe (1 mL or 3 mL Plastipak polypropylene syringe, Becton Dickinson) and a 21G×1.5 inch sterile needle (Becton Dickinson). The needle was removed from the filled syringe and the filled syringes were capped (Braun Combi-stoppers, polyethylene) and the final solution for administration was stored for up to 24 hours at 2 to 8° C.

As each dose was proportional to the concentration of CR845 in the vial (including placebo), the volume of study drug administered was dependent only upon the patient's estimated dry weight. Prepared syringes were blinded, and thus, the Investigator and study staff remained blinded to the treatment groups.

Part B (Days 3, 5, 10 and 12)

For Part B, the dose of 2.5 μg/kg (to be confirmed upon completion of Part A) or placebo was administered as a single I.V. bolus three times a week post-dialysis for two weeks.

Selection of Doses Used in the Study

The combined safety and PK data from an ascending Phase 1 dose ranging study of I.V. doses of 0.001 to 0.006 mg/kg in haemodialysis patients (Study CR845-CLIN1003) provided the basis for the selection of the doses of CR845 used in this study

Prior and Concomitant Medications

Prior medications were defined as those that the patient has taken during the 15 days prior to the Screening Visit through prior to the first dose of study drug on Day 1. Concomitant medications were medications that are taken from after the start of the first dose of study drug on Day 1 through the end of the study (i.e. follow up visit).

All prior and concomitant medications, including over-the-counter (OTC) medications used by patients during this study, were recorded in the appropriate source documents at each study visit and recorded on the appropriate page of the case report form (CRF).

Patients taking gabapentin, calcineurin inhibitors, opioids; antipsychotics; systemic or topical corticosteroids (other than otic or ophthalmic preparations); sedatives; hypnotics; antianxiety agents; SSRIs; or tricyclic antidepressants were required to remain on the same drugs at the same doses through the end of Week 2 unless a significant change in the patient's medical status necessitates a change.

Efficacy Assessment (Part B only)

The effect of CR845 on itch was measured by means of the following PROs:
Worst Itching on Visual Analog Scale (VAS);
Patient's Self-categorization of Pruritus Disease Severity;
Itch Medical Outcomes Study (MOS) sleep questionnaire;
Skindex-10;
Patient's treatment satisfaction.

Visual Analog Scale

Intensity of itch was measured using a 100-mm visual analog scale (VAS), in which the patient was asked to mark a line that represents the severity of their itch for the assessment time point, where “0=no itch” and “100=worst itch you can imagine”. Patients recorded their itch assessment scores on a worksheet under the direction of the site study staff, as defined below. The VAS has been widely utilized for evaluation of pruritus, including, uremic pruritus (See Refs^{3,4,5,6}).

Self-Categorization of Pruritus Disease Severity

Patients were asked to complete the Self-categorization of a Pruritus Disease Severity (PDS) questionnaire. Patients select a patient profile (A, B or C) that most closely resembles their profile, with profile A being the least affected by itch and profile C being the most affected by itch. Patients who classified themselves as Patient B or Patient C are eligible to qualify for inclusion in the study. This system of self-categorization of PDS was tested previously in a longitudinal study of uremic pruritus and found to correlate with both itch intensity and measurements from instruments evaluating quality of life (Ref.4).

Itch MOS Sleep Questionnaire

An Itch MOS sleep questionnaire was adapted from the Medical Outcomes Study (MOS) sleep survey in order to measure sleep disturbance as a result of nocturnal itching (Ref.4). For most questions, patients circled one of six numbers ranging from “1” (“all of the time”) to “6” (“none of the time”), indicating the frequency of various aspects of pruritus-related sleep disruption over the preceding week. Patients also estimated the average amount of sleep per night during the past week. The SLP-9 scoring method was utilized. The Itch MOS sleep questionnaire has been tested previously in a longitudinal study of uremic pruritus and found to correlate with both itch intensity as well as evaluating quality of life generally and to other measures of sleep and mood (Ref.4).

Skindex-10

Skindex-10 is a validated questionnaire developed specifically for uremic pruritus, for measurement of quality of life (Ref.4). Patients were asked to fill in one of seven bubbles (“0 [never bothered], 1, 2, 3, 4, 5, and 6 [always bothered]”) for each of ten questions. The total score is the sum of the numeric value of each answered question. The domain scores are sums of the following: disease domain (questions 1 to 3), mood/emotional distress domain (questions 4 to 6), and social functioning domain (questions 7 to 10). The Skindex-10 has been found to correlate with both itch intensity as well as other instruments evaluating quality of life (Ref.4).

Treatment Satisfaction

On Day 15 or at the time of early termination, patients were asked to provide an evaluation of the study drug by answering the following question: “How would you rate the study medication?”—

4 Excellent; 3 Very Good; 2 Good; 1 Fair or 0 Poor.

Safety Assessments

The safety assessments taken for each patient were the following:
Incidence and severity of AEs and SAEs
Physical examination;
Vital signs;
Oxygen saturation (Part A only);
12-lead ECG;
Clinical laboratory tests; and
Urine volume (Part A only).

Physical Examination

Physical examinations included, at minimum, an examination of the heart, lungs, abdomen, extremities, neurological system, and vascular system. Clinically significant abnormalities prior to administration of the first dose of study drug were reported as medical history and clinically significant new or worsening findings observed after the first dose of study drug are reported as adverse events (AEs).

Vital Signs

Vital signs included body temperature, HR, sitting or semi-recumbent BP, and RR. In Part A, oxygen saturation was also measured via pulse oximetry.

Measurements were repeated if a value was out of the reference range and additional measurements were taken at other times if judged to be clinically appropriate.

Measurement were taken at nominal time (±5 min).

Electrocardiogram

Twelve-lead ECGs were obtained and read locally by the Investigator or physician designee. Clinically significant abnormalities prior to administration of the first dose of study drug were reported as medical history and clinically significant new or worsening findings observed after the first dose of study drug were reported as AEs.

Clinical Laboratory Tests

The following clinical laboratory tests were performed and analyzed by one of the central laboratories. Processing and shipment of central laboratory samples was according to the Laboratory Manual protocols.

Hematology: hemoglobin, hematocrit, platelet count, white blood cell (WBC) count (including differential);

Serum Chemistry: total bilirubin, direct bilirubin (if total bilirubin is outside the ULN) and alkaline phosphatase alanine transaminase (ALT), aspartate aminotransferase (AST), glucose (non-fasting), serum creatinine, blood urea nitrogen (BUN), electrolytes (sodium, potassium, chloride, calcium and phosphorus), parathyroid hormone (PTH; Part B, on Day 1 only).

Additionally, serum sodium was measured in Part A

Urine Chemistry: Urine sodium and creatinine (Part A only, as applicable)

Serum Pregnancy: In women of childbearing potential

Follicle Stimulating Hormone: In women who had been amenorrheic for at least 1 year and were between 45 and 55 years of age to confirm that they were not of childbearing potential

Blood Hepcidin: Was analyzed in serum in Part A only.

24-Hour Urine Sample (Part A Only)

Patients were instructed to collect all urine over a 24-hour period for urine sodium, creatinine, and volume while in the clinical research unit. The collection started on the day prior to dialysis and ended prior to the start of dialysis the next day. Fluid intake was also measured during this period.

Adverse Events (AEs)

Definition of Adverse Events

A treatment-emergent AE (TEAE) is defined as any untoward medical occurrence in a patient administered a pharmaceutical product, and does not necessarily have a causal relationship with the treatment. An AE can be any unfavorable and unintended sign (e.g., including an abnormal laboratory finding), symptom, or disease temporally associated with the use of the study drug, whether or not it is considered to be study drug related.

This definition of a TEAE includes any newly occurring event or previous condition that has increased in severity or frequency since the administration of study drug.

Abnormal laboratory tests at Screening or before the administration of the study drug that were assessed as clinically significant were not reportable as AEs. Clinically significant abnormalities prior to administration of the first dose of study drug were reported as medical history, unless they were expected findings from medical history that had already been reported (e.g., elevated PTH in a patient with a medical history of hyperparathyroidism).

AEs resulting from concurrent illnesses, reactions to concurrent illnesses, reactions to concurrent medications, or progression of disease states were also be recorded. In order to avoid vague, ambiguous, or colloquial expressions, AEs were recorded in standard medical terminology rather than the patient's own words. Signs and symptoms were reported individually unless, in the judgment of the Investigator, they could be grouped under a widely accepted inclusive term (e.g., gastroenteritis in lieu of abdominal pain, nausea, vomiting, and diarrhea).

An abnormal result of a diagnostic procedure following administration of the study drug is captured as an AE if the finding meets the following criteria:
Results in study withdrawal;
Is associated with signs or symptoms;
Is considered by the Investigator to be of clinical significance.

The underlying diagnosis as captured as the AE, not the procedure itself.

Overdose was defined as an accidental or intentional exposure to study drug at a dose higher than specified in the protocol or higher than known therapeutic dose. Any overdose of study drug was reportable as a protocol deviation. Any overdose associated with clinical signs or symptoms, were captured as AEs.

Pharmacokinetic Evaluation (Part A only)

For PK analysis, approximately 4 mL of blood was collected from the predialyzer (arterial) line at the following times:

Day 1: Within 5 min. prior to starting dialysis; and within 10 min. following the end of dialysis (i.e. from return of blood to patent).

At 5 mins., 15 mins. and 30 mins. and at 1, 2, 3, 4, 6, 8 12 and 24 hours after injection of CR845; (i.e. t=0). A ±1 minute window is allowed for PK blood draws up to 30 min (inclusive), a ±5 minute window was allowed for the remainder of the PK assessments.

Day 3: Within 5 min. prior to starting dialysis; Day 5: Within 5 min. prior to starting dialysis; and within 10 min. following the end of dialysis (i.e. from return of blood to patent).

At 5 mins., 15 mins. and 30 mins. and at 1, 2, 3, 4, 6, 8 12 and 24 hours after injection of CR845; (i.e. t=0). A ±1 minute window was allowed for PK blood draws up to 30 min (inclusive), a ±5 minute window was allowed for the remainder of the PK assessments.

Day 8: Within 5 min. prior to starting dialysis; and within 10 min. following the end of dialysis (i.e. from return of blood to patent).

Plasma samples were analyzed for CR845 using liquid chromatography with tandem mass spectrometric detection (LC/MS/MS) according to validated analytical methods. Blood samples were collected in 4 mL lavender-top vacutainer tubes containing K2EDTA as the anticoagulant. The total blood volume collected for CR845 PK sampling was approximately 108 mL. Blood samples were placed on ice immediately after collection and remain on ice throughout processing. Blood samples were then centrifuged according to standard phlebotomy sample collection procedures. The study drug is stable in whole blood on ice for at least 1 hour, so sample processing was completed no later than 1 hour from the collection time. The resulting plasma was transferred in aliquots of approximately 1 mL each into two appropriately labeled polypropylene screw-cap tubes. All sample collection and freezing tubes were to be clearly labeled. Plasma samples are frozen at −70° C. or below. The samples were frozen within 1 hour of collection and remain frozen until assayed. The actual time of collection was recorded. Samples were shipped frozen on dry ice to an analytical laboratory.

Statistical Methods

General Considerations

All statistical tests are performed at the α=0.05 significance level using one-sided tests, unless otherwise noted.

Interim Analysis

An unblinded interim analysis was performed following the completion of Part A. There was no interim analysis for the Part B of the study.

Analysis Populations

Four analysis populations were defined as follows:
Safety Population (Parts A and B): All patients who were randomized and had received any study drug at any time during the study comprise the safety population. Within the safety population, treatment assignment was made on an “as treated” basis.
Pharmacokinetic Evaluable Population (Part A only): The PK evaluable population included all patients who received CR845 and have sufficient plasma concentrations for PK analysis are included in the PK population.
Modified Intent-To-Treat (MITT) Population (Part B only): All patients who were randomized and received at least one dose of the study drug, and at least one post-baseline efficacy assessment constituted the MITT population.

Assignment of patients to treatment condition was made on an “as randomized” basis.
Per Protocol (PP) Population (Part B only): All patients who met the MITT population criteria without major protocol deviations adversely affecting the efficacy data analysis constituted the PP population.

The MITT population was used for the summaries of protocol deviations, demographics, and baseline disease characteristics. The safety population was used for the summaries of all safety assessments. The MITT and PP populations were used for the analysis and summaries of efficacy endpoints. The PK evaluable population was used for all PK data summary.

Data from placebo-treated patients from the different dosing groups in Part A were combined for analysis and reporting.

Patient Disposition

The number of patients randomized, completed, or discontinued from the study, along with the reason for discontinuation, was presented overall and by treatment group. Patient count by analysis population was also tabulated.

Efficacy Analysis

Primary Efficacy Endpoints (Part B)

The primary efficacy endpoint was the change from baseline to the average of the Week 2 worst itching VAS. The baseline for the VAS score is defined as the average of all respective assessments during the Run-In period. The mean of the last 8 VAS scores from Week 2 was used for the comparison.

Mixed model for repeated measures (MMRM) analysis was applied with the daily worst itching VAS scores during the 2-week treatment period serving as the dependent variable. The model included the baseline VAS score as covariate, treatment, week (Weeks 1 and 2), day within week (Days 1 through 7)), the treatment by week interaction as fixed effects, and patient identification as the unit for repeated measures. The between treatment difference was estimated as the simple contrast at Week 2 in the model treatment parameter. The primary analysis was based on the MITT population and the sensitivity analysis was based on the per protocol population.

To explore the robustness of the results, other statistical method, such as analysis of covariance (ANCOVA) or missing data imputation was also applied to this endpoint.

Other Efficacy Endpoints and Exploratory Endpoints (Parts A and B)

Endpoints of a continuous data nature were analyzed similarly as for the primary endpoint. Endpoints of ordinal or categorical data nature are analyzed using generalized linear model (e.g., logistical regression) or non-parametric test.

Pharmacokinetic Analysis (Part A)

Raw serum values and PK parameters for CR845 were summarized using appropriate descriptive statistics. Plasma concentrations were summarized descriptively and graphically by nominal time. Pharmacokinetic parameters (half-life, Cmax, Tmax, AUC, Vd, etc) were calculated from the plasma concentration data using validated software such WinNonlin. Individual plasma CR845 concentrations were listed and plotted by patient. Statistical tests were applied to raw values and PK parameters of each sample to obtain a full PK profile analysis.

Ethics

The study was conducted in accordance with ethical principles founded in the Declaration of Helsinki (Edinburgh 2000) and all accepted amendments, the ICH principles of GCP (including archiving of essential study documents), and the applicable regulations of the country in which the study is conducted. The protocol, the Investigator's informed consent document, and related patient information and recruitment materials were reviewed and approved by an Institutional Review Board (IRB) or Independent Ethics Committee (IEC), before the start of the study.

Good Clinical Practices (GCP)

The study was conducted in accordance with the ICH for GCP and the appropriate regulatory requirement(s).

Institutional Review Board (IRB)

The IRB reviewed and approved the protocol, ICF, and related patient information and recruitment materials before the start of the study.

Results of Clinical Trial

The above-described randomized, double-blind, placebo controlled study was run with sixty-five patients receiving a dose of either CR845 or placebo after each dialysis session, three times per week. The trial was run in twenty one clinical centers in the United States. Thirty-three of the sixty-five patients in the trial received CR845, the remaining thirty-two patients received placebo. Three patients terminated early and did not complete the study.

Fifty-seven patients, thirty receiving CR845 and twenty-seven receiving placebo, complied with the protocol. Fifty-six patients received all six planned doses of CR845 (twenty-six patients) or placebo (thirty patients). One patient received five of the six doses of placebo and two patients received five of the six doses of CR845. One patient received four doses and two patients received three of the six planned doses of placebo. One patient received two of the six planned doses of placebo, and one patient received only one of the six planned doses of CR845.

FIG. 24 shows the pharmacokinetic profile for each of the 0.5 mg/kg, 1.0 mg/kg and 2.5 mg/kg doses of CR845 over days two and five of the trial and tracks the level of CR845 in patient blood before and after dialysis. Surprisingly, the area under the curve (AUC), an indicator of bioavailability, is ten-fold greater in these dialysis patients than in health volunteers. This suggests that loss of kidney function extends the half-life of CR845 in the blood of dialysis patients and makes patients suffering from chronic kidney dysfunction well suited for CR845 treatment.

The change in itch from baseline determined pre-trial on a visual analog scale (VAS) as indicated by placebo-treated dialysis patients and dialysis patients treated with 1 ug/kg CR845 is shown in FIG. 25. CR845 treatment resulted in a 54% reduction in itch, which was highly significant at the p=0.016 level.

FIG. 26 shows the change in itch in placebo-treated and CR845-treated dialysis patients with end stage renal disease (ESRD) during week prior to clinical trial (the so-called run-in period), and at week 1 and week 2 of the CR845-treatment trial. The level of itch determined on a visual analog scale (VAS) in CR845-treated patients was 62% lower than in placebo-treated patients, significant at the p<0.05 level.

The average of “worst itching” (i.e. the highest patient-scored itch) over daytime and night time as assessed by a visual analog scale (VAS) in CR845-treated dialysis patients was monitored over the 15 days of the trial (see FIG. 27). The reduction of itch intensity in dialysis patients treated with CR845 continued throughout the two week treatment period. Dialysis patients who received placebo experienced an initial improvement in itching in the first week, which reached a plateau in the second week as might be expected for a placebo effect. Worst daytime and night time itch levels are shown in FIGS. 28A and 28B, respectively, for CR845-treated and placebo treated dialysis patients over the three week period from the run-in week prior to initiation of the trial, throughout the two weeks of the trial. The average itch reduction over both daytime and night time was found to be 48% from baseline. Significantly, the observed 75% reduction in worst night time itch was highly significant (p=0.007), and daytime reduction of 51% was also significant (p=0.03).

Quality of life effects due to treatment with CR845 was assessed by the Skindex-10 questionnaire (see above) divided into three domains: The first domain was the “Disease Domain” consisting of three questions; the “Mood/Emotional Distress” domain also consisting of three questions; and the “Social Functioning Domain” consisting of four questions.

Each of the ten questions ask “during the past WEEK, how often have you been bothered by” the following questions on a scale of 0=Never bothered to 6=always bothered:

I. “Disease Domain”
1. Your itching.
2. The persistence/recurrence of your itching.
3. The appearance of your skin from scratching.

II “Mood/Emotional Distress”
4. Frustration about your itching.
5. Being annoyed about your itching.
6. Feeling depressed about your itching.

III. “Social Functioning”
7. Feeling embarrassed about your itching.
8. The effects of your itching on your interactions with others.
9. The effects of your itching on your desire to be with people.
10. The effects of your itching making it hard to work or do what you enjoy.

The overall change in Skindex scores from baseline determined at day 1 for the placebo-treated patients and the patients treated with 1 ug/kg CR845 from day 1 through day 15 of the trial is shown in FIG. 29. Treatment with 1 ug/kg CR845 resulted in a 71% reduction in Skindex-10 scores compared with placebo with a significance of p=0.031.

Skindex-10 scores for each if the three domains: Disease; Mood/Emotional Distress; and Social Functioning; for CR845-treated and placebo-treated dialysis patients are shown in FIG. 30. In each domain, CR845-treatment resulted in a trend toward lower Skindex-10 scores (i.e. a surrogate measure for improved quality of life) in all aspects represented by the three tested domains. Mood/emotional distress reduction was significant (p=0.046).

The itch MOS sleep index (SLP-9: See above) was used to track trends in the effect of CR845 treatment on itch-related sleep disturbance. FIG. 31 shows the compiled results of the SLP-9 score over the 15 day treatment period of the trial for placebo-treated and CR845-treated dialysis patients. The results show a 62% reduction (i.e. improvement) in sleep as represented by SLP-9 score associated with CR845-treatment as compared with placebo.

In this clinical trial, I.V. CR845 was shown to be safe and well tolerated with no CR845-related serious adverse events (AEs) reported. The most common AEs were transient numbness and dizziness, with no episodes of CNS side effects (e.g., dysphoria and hallucinations).

### REFERENCES

1. Investigator's Brochure for CR845. Edition No. 6, June 2014, Cara Therapeutics, Inc.
2. Wikström B, Gellert R, Ladefoged S D, Danda Y, Akai M, Ide K, Ogasawara M, Kawashima Y, Ueno K, Mori A, Ueno Y. Kappa-opioid system in uremic pruritus: multicenter, randomized, double-blind, placebo-controlled clinical studies. J. Am. Soc. Nephrol. 2005; 16(12):3742-47.
3. Mathur V S, Lindberg J, Germain M, Block G, Tumlin J, Smith M, Grewal M, McGuire D. A longitudinal study of uremic pruritus in haemodialysis patients. Clin. J. Am. Soc. Nephrol. 2010; 5:1410-1419.
4. Kumagai H, Ebata T, Takamori K, Muramatsu, T, Nakamoto H, Suzuki H. Effect of a novel kappa-receptor agonist, nalfurafine hydrochloride, on severe itch in 337 haemodialysis patients: a Phase III, randomized, double-blind, placebo-controlled study. NDT. 2009; April; 25(4):1251-1257.
5. Ständer S, Richter L, Osada N, Metze D. Hydroxyethyl Starch-induced Pruritus: Clinical Characteristics and Influence of Dose, Molecular Weight and Substitution. Acta Derm Venereol. 2013 Sep. 16.
6. Pisoni R L, Wikstrom B, Elder S J, Akizawa T, Asano Y, Keen M L, Mendelssohn D C, Young E W, and Port F K. Pruritus in haemodialysis patients: international results from the Dialysis Outcomes and Practice Patterns Study (DOPPS). NDT. 2006; 21: 3495-3505.

The specifications of each of the U.S. patents and published patent applications, and the texts of the literature references cited in this specification are herein incorporated by reference in their entireties. In the event that any definition or description contained found in one or more of these references is in conflict with the corresponding definition or description herein, then the definition or description disclosed herein is intended.

The examples provided herein are for illustration purposes only and are not intended to limit the scope of the invention, the full breadth of which will be readily recognized by those of skill in the art.

1. A method of prevention, inhibition or treatment of uremic pruritus in a patient, the method comprising administering an effective amount of a kappa opioid receptor agonist comprising a synthetic peptide amide to the patient, wherein the patient is undergoing a dialysis regimen and the kappa opioid receptor agonist comprising the synthetic peptide amide is administered on at least one of the days in which dialysis occurs.

2. The method according to claim 1, wherein the kappa opioid receptor agonist comprising a synthetic peptide amide is administered on at least two of the days in which dialysis occurs.

3. The method according to claim 1, wherein the kappa opioid receptor agonist comprising a synthetic peptide amide is administered three times per week for at least one week in which dialysis occurs.

4. The method according to claim 1, wherein the administration of the kappa opioid receptor agonist comprising a synthetic peptide amide is within one hour after dialysis.

5. The method according to claim 1, wherein the administration of the kappa opioid receptor agonist comprising a synthetic peptide amide is by intravenous injection.

6. The method according to claim 5, wherein the intravenous injection is by an i.v. bolus injection.

7. The method according to claim 1, wherein the effective amount of the kappa opioid receptor agonist comprising a synthetic peptide amide administered is estimated from the patient's dry weight.

8. The method according to claim 7, wherein the effective amount of the kappa opioid receptor agonist comprising a synthetic peptide amide administered is from about 0.1 μg/kg of the patient's dry weight to about 5.0 μg/kg of the patient's dry weight.

9. The method according to claim 8, wherein the effective amount of the kappa opioid receptor agonist comprising a synthetic peptide amide administered is from about 0.5 μg/kg of the patient's dry weight to about 2.5 μg/kg of the patient's dry weight.

10. The method according to claim 9, wherein the effective amount of the kappa opioid receptor agonist comprising a synthetic peptide amide administered is about 2.5 μg/kg of the patient's dry weight.

11. A method of prevention, inhibition or treatment of an adverse symptom associated with dialysis in a patient undergoing dialysis, the method comprising administering an effective amount of a kappa opioid receptor agonist comprising a synthetic peptide amide to the patient undergoing dialysis.

12. The method according to claim 11, wherein the adverse symptom associated with dialysis is selected from the group consisting of uremic pruritus, sleep disruption, and mood alteration.

13. The method according to claim 12, wherein the adverse symptom associated with dialysis is sleep disruption.

14. The method according to claim 13, wherein the adverse symptom associated with dialysis is sleep disruption is due to pruritus.

15. The method according to claim 12, wherein the adverse symptom associated with dialysis is mood alteration, and the mood alteration is depression.

16. The method according to claim 1, wherein the kappa opioid receptor agonist comprising a synthetic peptide amide is CR845: 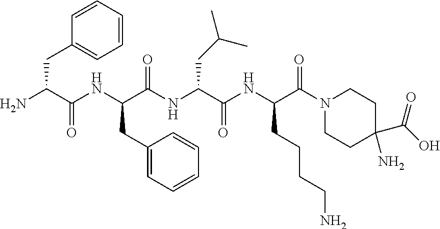 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]
CR845: D-Phe-D-Phe-D-Leu-D-Lys-[ω(4-aminopiperidine-4-carboxylic acid)]-OH

17. The method according to claim 16, wherein the kappa opioid receptor agonist is administered on at least two days in which the dialysis occurs.

18. The method according to claim 16, wherein the kappa opioid receptor agonist is administered three times per week for at least one week.

19. The method according to claim 16, wherein at least one administration of the kappa opioid receptor agonist is within one hour after dialysis.

20. The method according to claim 16, wherein at least one administration of the kappa opioid receptor agonist is by intravenous injection.
